(19) United States
(12) Patent Application Publication
(10) Pub. No.: US 2011/0201622 A1
(43) Pub. Date: Aug. 18, 2011

(54) Solid Forms Comprising A Cyclopropyl Amide Derivative

(76) Inventor: Craig D. COLLINS, Wilmington, DE (US)

(21) Appl. No.: 13/029,351

(22) Filed: Feb. 17, 2011

Related U.S. Application Data
(60) Provisional application No. 61/305,581, filed on Feb. 18, 2010.

(51) Int. Cl. A61K 31/495; C07D 241/04; A61P 3/04; A61P 25/18; A61P 25/28

(52) U.S. Cl. 514/255.01; 544/391

(57) ABSTRACT

This disclosure relates to at least one solid form of 4-{(1S, 2S)-2-[((R)-4-cyclobutyl-2-methylpiperazin-1-yl)carbonyl]-cyclopropyl}-benzamide. This disclosure also relates to at least one pharmaceutical composition comprising at least one solid form described herein, methods of using the solid forms and pharmaceutical compositions comprised thereof, and processes of manufacturing the solid forms.

### CROSS REFERENCE TO RELATED APPLICATIONS

This application claims the benefit of U.S. Provisional Application No. 61/305,581, filed Feb. 18, 2010, the entirety of which is incorporated herein by reference.

This disclosure relates to at least one solid form of 4-{(1S, 2S)-2-[((R)-4-cyclobutyl-2-methylpiperazin-1-yl)carbonyl]-cyclopropyl}-benzamide. This disclosure also relates to at least one pharmaceutical composition comprising at least one solid form described herein, methods of using the solid forms and pharmaceutical compositions comprised thereof, and processes of manufacturing the solid forms.

In the formulation of drug compositions, it is desirable for the active drug substance to be in a form in which it can be conveniently handled and processed. This is of importance not only from the viewpoint of obtaining a commercially viable manufacturing process, but also from the viewpoint of subsequent manufacture of pharmaceutical formulations comprising the active drug substance. Further, in the manufacture of drug compositions, it is desirable that a reliable, reproducible and constant plasma concentration profile of drug is provided following administration to a patient.

Chemical stability, solid-state stability, and shelf life of the active ingredients are also desirable factors. The drug substance, and compositions containing it, should preferably be capable of being effectively stored over appreciable periods of time, without exhibiting a significant change in the active component's physico-chemical characteristics (e.g., its chemical composition, density, hygroscopicity and solubility). Moreover, it is desirable to provide a drug substance in a form that is as chemically pure as possible.

It is also desirable to provide advantageous solid form(s) of a drug, which in some cases may afford particular desirable characteristics, such as, for example, ease of handling, ease of preparation of suitable pharmaceutical formulations, and a more reliable solubility profile.

There remains a need for solid form(s) of 4-{(1S, 2S)-2-[((R)-4-Cyclobutyl-2-methylpiperazin-1-yl)carbonyl]-cyclopropyl}-benzamide, which have one or more advantageous physical properties, such as, for example, stability, solubility, processability, and bioavailability.

### BRIEF DESCRIPTION OF DRAWINGS

FIG. 1 shows an X-ray powder diffraction (XRPD) pattern for Form I of Compound I.

FIG. 2 shows a differential scanning calorimetry (DSC) thermogram for Form I of Compound I.

FIG. 3 shows a thermal gravimetric analysis (TGA) thermogram for Form I of Compound I.

FIG. 4 shows a dynamic vapor sorption (DVS) isotherm plot for Form I of Compound I.

FIG. 5 shows a ¹³C cross polarization magic angle spinning (CPMAS) solid-state nuclear magnetic resonance (SS-NMR) spectrum for Form I of Compound I.

FIG. 6 shows a Fourier Transform Infrared (FT-IR) spectrum (top) and FT-Raman spectrum (bottom) for Form I of Compound I.

Embodiments herein relate to solid forms of “Compound I”, which is described by the chemical name 4-{(1S, 2S)-2-[((R)-4-cyclobutyl-2-methylpiperazin-1-yl)carbonyl]-cyclopropyl}-benzamide and the chemical structure (I), shown below:

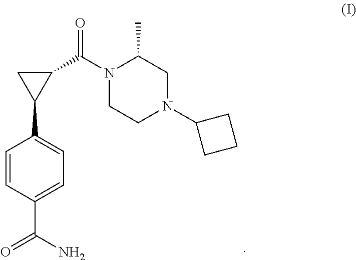

Further embodiments described herein relate to at least one pharmaceutical composition comprising at least one solid form described herein, methods of using the solid forms and pharmaceutical compositions described herein, and processes of manufacturing the solid forms.

One embodiment provides a solid form of Compound I that is substantially crystalline. The term “substantially crystalline” includes crystallinity greater than 20%, greater than 30%, greater than 40%, greater than 50%, greater than 60%, greater than 70%, greater than 80%, greater than 90%, greater than 95%, greater than 97%, greater than 98%, or greater than 99% on a weight basis.

Another embodiment provides a solid form of Compound I that is partially crystalline. The term “partially crystalline” includes crystallinity that is less than 20%, less than 10%, or less than 5% by weight. The degree (%) of crystallinity may be determined by the skilled person using a variety of techiniques, including, but not limited to, for example, XRPD, SS-NMR spectroscopy, FT-IR spectroscopy, FT-Raman spectroscopy, DSC thermoanalysis, TGA analysis, microcalorimetry, and DVS analysis.

Yet another embodiment provides a solid form of Compound I that is substantially pure. In specific embodiments, the term “substantially pure” includes samples of a solid form that are greater than 50% chemically pure, greater than 60% chemically pure, greater than 70% chemically pure, greater than 80% chemically pure, greater than 90% chemically pure, greater than 95% chemically pure, greater than 98% chemically pure, or greater than 99% chemically pure Compound I on a weight basis with regard to chemical compounds other than Compound I. The degree (%) of chemical purity may be determined by the skilled person using a variety of techiniques, including, but not limited to, for example, NMR spectroscopy, high performance liquid chromatography (HPLC), mass spectrometry (MS), and elemental analysis (e.g., combustion analysis). In specific embodiments, the term “substantially pure” includes samples of a selected solid form that are greater than 50% physically pure, greater than 60% physically pure, greater than 70% physically pure, greater than 80% physically pure, greater than 90% physically pure, greater than 95% physically pure, greater than 98% physically pure, or greater than 99% physically pure solid form on a weight basis with regard to solid forms other than the selected solid form (e.g., other crystal forms or amorphous forms). The degree (%) of physical purity may be determined by the skilled person using a variety of techiniques, including, but not limited to, for example XRPD, SS-NMR spectroscopy, FT-IR spectroscopy, FT-Raman spectroscopy, DSC thermoanalysis, TGA analysis, microcalorimetry, and DVS analysis.

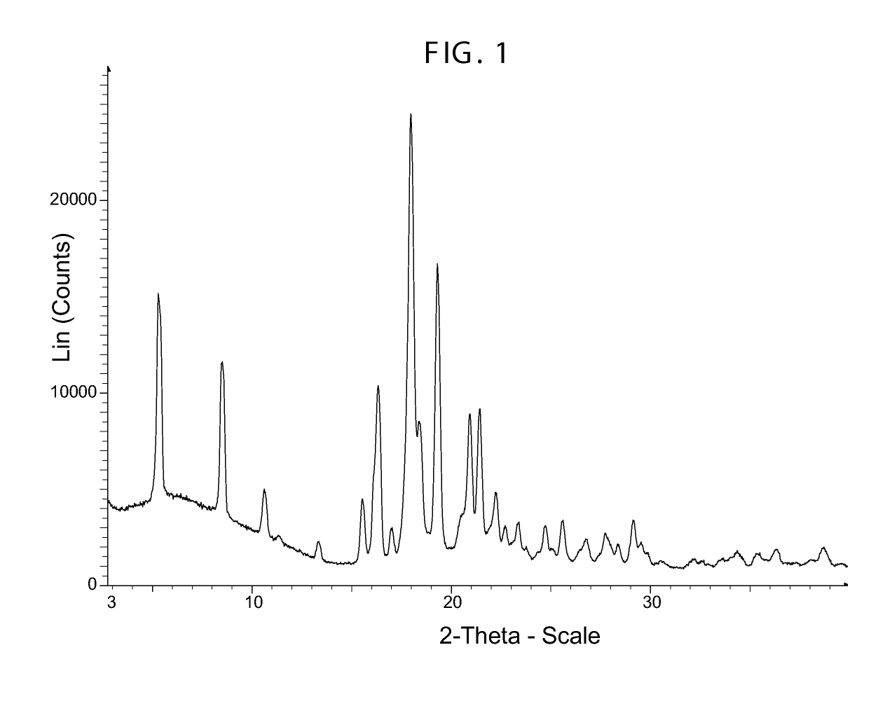

Still another embodiment provides a solid form that is Form I of Compound I. An XRPD pattern, DSC thermogram, TGA thermogram, DVS isotherm plot, SS-NMR s spectrum, FT-IR spectrum, and FT-Raman spectrum for representative Form I material are shown in FIGS. 1-6. In particular embodiments, Form I of Compound I is substantially crystalline. In other particular embodiments, Form I of Compound I is substantially pure. In yet other particular embodiments, Form I of Compound I is substantially crystalline and substantially pure.

Yet still another embodiment relates to Form I of Compound I that has an XRPD pattern comprising peaks essentially as defined in Table 1.

It is known in the art that an XRPD pattern may be obtained which has one or more measurement errors depending on measurement conditions (such as equipment, sample preparation or machine used). In particular, it is generally known is that intensities in an XRPD pattern may fluctuate depending on measurement conditions and sample preparation. For example, persons skilled in the art of XRPD will realise that the relative intensities of peaks may vary according to the orientation of the sample under test and on the type and setting of the instrument used. The skilled person will also realise that the position of reflections can be affected by the precise height at which the sample sits in the diffractometer and the zero calibration of the diffractometer. The surface planarity of the sample may also have a small effect. As a result, a person skilled in the art will appreciate that the diffraction pattern data presented herein is not to be construed as absolute and any crystalline form that provides an XRPD pattern substantially identical to those disclosed herein fall within the scope of the present disclosure. The person of skill in the art further appreciates that XRPD 2θ values may vary with a reasonable range, e.g., in the range ±0.1°2θ to ±0.2°2θ. Principles of XRPD are described in publications, such as, for example, Giacovazzo, C. et al. (1995), Fundamentals of Crystallography, Oxford University Press; Jenkins, R. and Snyder, R. L. (1996), Introduction to X-Ray Powder Diffractometry, John Wiley & Sons, New York; and Klug, H. P. & Alexander, L. E. (1974), X-ray Diffraction Procedures, John Wiley and Sons, New York.

A further embodiment relates to Form I of Compound I that has an XRPD pattern essentially as depicted in FIG. 1.

A yet further embodiment relates to Form I of Compound I that has an XRPD pattern comprising peaks at any one, two, three, four, five, six, seven, eight, nine or ten of the following positions: about 5.3, about 8.5, about 10.6, about 15.5, about 16.3, about 18.0, about 18.4, about 19.3, about 20.9, about 21.4 °2θ, when measured using radiation with a wavelength of about 1.54 angstroms.

A still further embodiment relates to Form I of Compound I that has an XRPD pattern comprising peaks at any one of the following positions: about 5.3, about 8.5, about 10.6, about 15.5, about 16.3, about 18.0, about 18.4, about 19.3, about 20.9, about 21.4 °2θ, when measured using radiation with a wavelength of about 1.54 angstroms.

An even further embodiment relates to Form I of Compound I that has an XRPD pattern comprising peaks at any two of the following positions: about 5.3, about 8.5, about 10.6, about 15.5, about 16.3, about 18.0, about 18.4, about 19.3, about 20.9, about 21.4 °2θ, when measured using radiation with a wavelength of is about 1.54 angstroms.

Another embodiment relates to Form I of Compound I that has an XRPD pattern comprising peaks at any three of the following positions: about 5.3, about 8.5, about 10.6, about 15.5, about 16.3, about 18.0, about 18.4, about 19.3, about 20.9, about 21.4 °2θ, when measured using radiation with a wavelength of about 1.54 angstroms.

Yet another embodiment relates to Form I of Compound I that has an XRPD pattern comprising peaks at any four of the following positions: about 5.3, about 8.5, about 10.6, about 15.5, about 16.3, about 18.0, about 18.4, about 19.3, about 20.9, about 21.4 °2θ, when measured using radiation with a wavelength of about 1.54 angstroms.

Still yet another embodiment relates to Form I of Compound I that has an XRPD pattern comprising peaks at any five of the following positions: about 5.3, about 8.5, about 10.6, about 15.5, about 16.3, about 18.0, about 18.4, about 19.3, about 20.9, about 21.4 °2θ, when measured using radiation with a wavelength of about 1.54 angstroms.

A still yet further embodiment relates to Form I of Compound I that has an XRPD pattern comprising peaks at any six of the following positions: about 5.3, about 8.5, about 10.6, about 15.5, about 16.3, about 18.0, about 18.4, about 19.3, about 20.9, about 21.4 °2θ, when measured using radiation with a wavelength of about 1.54 angstroms.

An even yet further embodiment relates to Form I of Compound I that has an XRPD pattern comprising peaks at any seven of the following positions: about 5.3, about 8.5, about 10.6, about 15.5, about 16.3, about 18.0, about 18.4, about 19.3, about 20.9, about 21.4 °2θ, when measured using radiation with a wavelength of about 1.54 angstroms.

A further embodiment relates to Form I of Compound I that has an XRPD pattern comprising peaks at any eight of the following positions: about 5.3, about 8.5, about 10.6, about 15.5, about 16.3, about 18.0, about 18.4, about 19.3, about 20.9, about 21.4 °2θ, when measured using radiation with a wavelength of about 1.54 angstroms.

A still further embodiment relates to Form I of Compound I that has an XRPD pattern comprising peaks at any nine of the following positions: about 5.3, about 8.5, is about 10.6, about 15.5, about 16.3, about 18.0, about 18.4, about 19.3, about 20.9, about 21.4 °2θ, when measured using radiation with a wavelength of about 1.54 angstroms.

An even further embodiment relates to Form I of Compound I that has an XRPD pattern comprising peaks at the following ten positions: about 5.3, about 8.5, about 10.6, about 15.5, about 16.3, about 18.0, about 18.4, about 19.3, about 20.9, about 21.4 °2θ, when measured using radiation with a wavelength of about 1.54 angstroms.

In another embodiment, Form I of Compound I has an XRPD pattern comprising at least one peak selected from about 5.3, about 8.5, and about 18.0 °2θ, when measured using radiation with a wavelength of about 1.54 angstroms.

In a further embodiment, Form I of Compound I has an XRPD pattern comprising at least two peaks selected from about 5.3, about 8.5, and about 18.0 °2θ, when measured using radiation with a wavelength of about 1.54 angstroms.

In one embodiment, Form I of Compound I has an XRPD pattern comprising a peak at about 18.0 °2θ, when measured using radiation with a wavelength of about 1.54 angstroms.

In a further embodiment, Form I of Compound I that has an XRPD pattern comprising peaks at about 16.3 and about 19.3 °2θ, when measured using radiation with a wavelength of about 1.54 angstroms.

In another embodiment, Form I of Compound I has an XRPD pattern comprising peaks at about 5.3, about 18.0, and about 19.3 °2θ, when measured using radiation with a wavelength of about 1.54 angstroms.

In still another embodiment, Form I of Compound I has an XRPD pattern comprising peaks at about 5.3, about 8.5, and about 18.0 °2θ, when measured using radiation with a wavelength of about 1.54 angstroms.

In yet still another embodiment, Form I of Compound I has an XRPD pattern comprising peaks at about 5.3, about 8.5, about 18.0, and about 19.3 °2θ, when measured using radiation with a wavelength of about 1.54 angstroms.

In a still further embodiment, Form I of Compound I has an XRPD pattern comprising peaks at about 5.3, about 8.5, about 16.3, about 18.0, and about 19.3 °2θ, when measured using radiation with a wavelength of about 1.54 angstroms.

In an even still further embodiment, Form I of Compound I has an XRPD pattern comprising peaks at about 5.3, about 8.5, about 16.3, about 18.0, about 19.3, is about 20.9, and about 21.4 °2θ, when measured using radiation with a wavelength of about 1.54 angstroms.

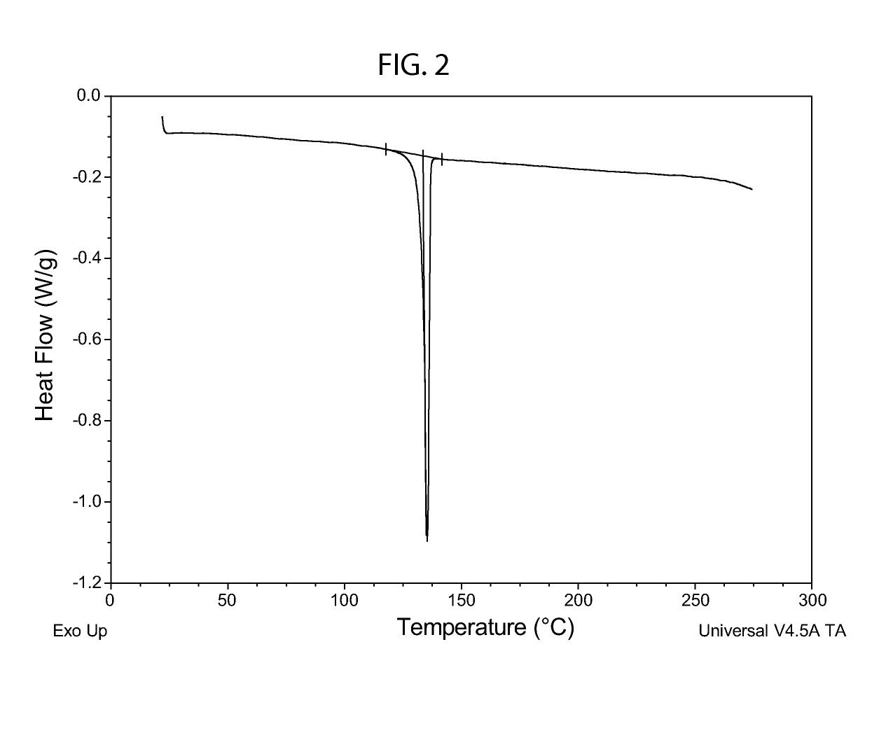

Another embodiment relates to Form I of Compound I that has a DSC thermogram essentially as depicted in FIG. 2.

It is well known that the DSC onset and peak temperatures as well as energy values may vary due to, for example, the purity of the sample and sample size and due to instrumental parameters, especially the temperature scan rate. Hence the DSC data presented are not to be taken as absolute values. A person skilled in the art can set up instrumental parameters for a Differential scanning calorimeter so that data comparable to the data presented here can be collected according to standard methods, for example those described in Höhne, G. W. H. et al (1996), Differential Scanning calorimetry, Springer, Berlin.

In another embodiment, Form I of Compound I has a DSC thermogram comprising an endothermic event with an onset temperature of about 133.5° C.

In still another embodiment, Form I of Compound I has a DSC thermogram comprising an endothermic event with a peak temperature of about 135.3° C.

In yet still another embodiment, Form I of Compound I has a DSC thermogram exhibiting no significant endothermic events between about 20° C. and about 130° C.

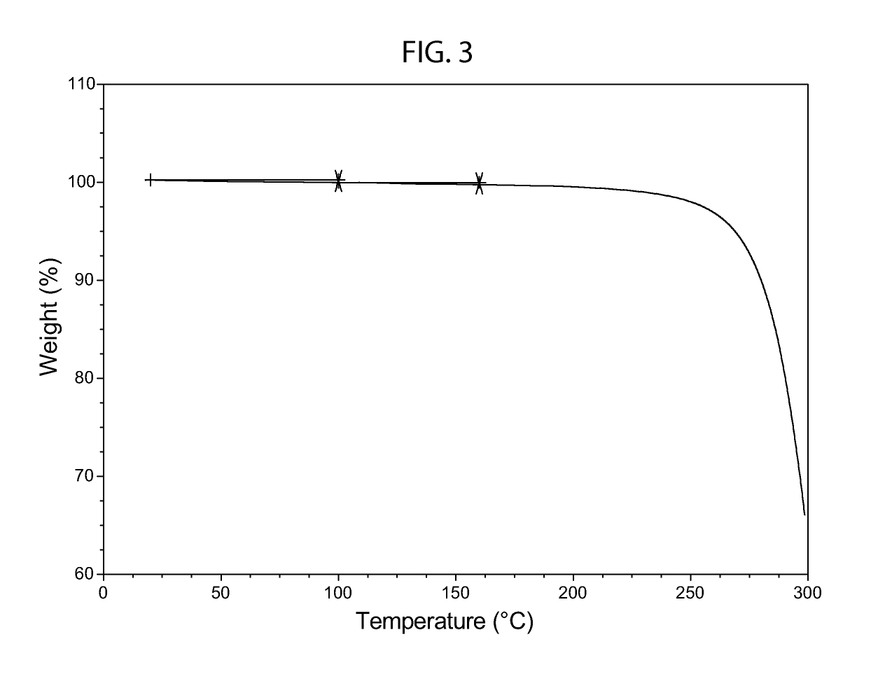

A further embodiment relates to Form I of Compound I that has a TGA thermogram essentially as depicted in FIG. 3.

It is well known that the TGA trace may vary due to, for example, the sample size and due to instrumental parameters, especially the temperature scan rate. Hence the TGA data presented are not to be taken as absolute values.

In one embodiment, Form I of Compound I has a TGA thermogram comprising a weight loss of less than about 1% (e.g., less than about 0.75%, less than about 0.5%, less than about 0.25%, or about 0%) of the total weight of the sample when heated from about 20° C. to about 100° C.

In a further embodiment, Form I of Compound I has a TGA thermogram comprising a weight loss of less than about 1% (e.g., less than about 0.75%, less than about 0.5%, less than about 0.25%, or about 0%) of the total weight of the sample when heated from about 100° C. to about 160° C.

In another embodiment, Form I of Compound I does not contain substantial is amounts of solvent (e.g., water, ethyl acetate (EtOAc), and/or acetonitrile (ACN)). In particular embodiments, Form I of Compound I contains less than about 3%, less than about 2%, less than about 1%, less than about 0.75%, less than about 0.5%, less than about 0.25%, or less than about 0.1% solvent (e.g., water, EtOAc, and/or ACN) on a weight basis.

In yet another embodiment, Form I of Compound I is not solvated.

In still another embodiment, Form I of Compound I is anhydrous.

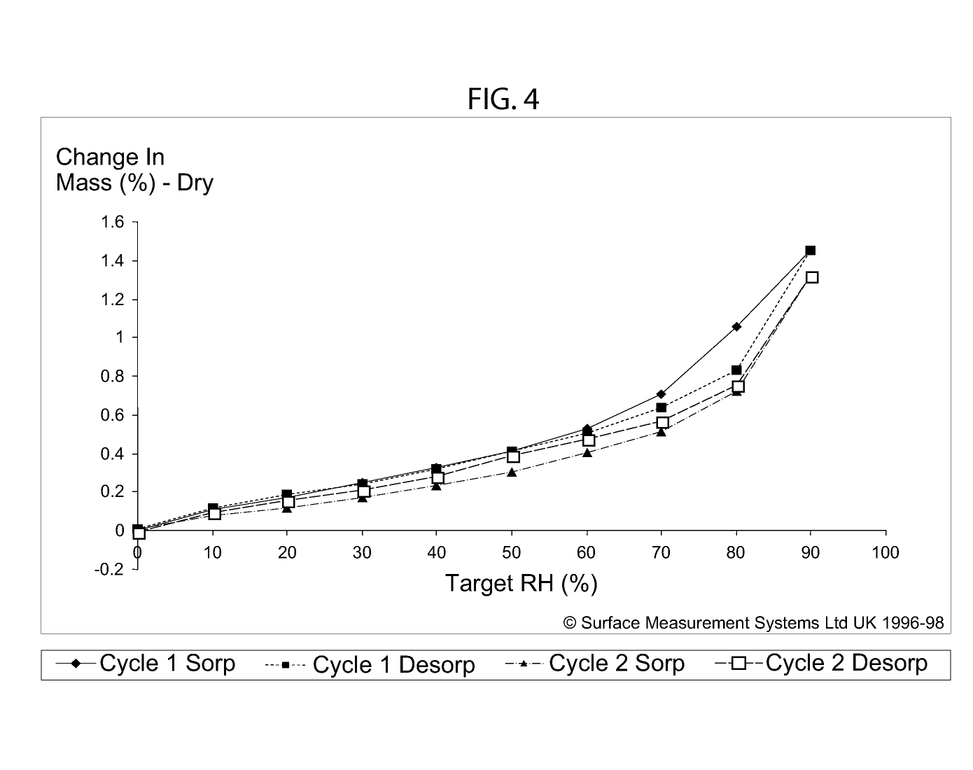

A still further embodiment relates to Form I of Compound I that has a DVS isotherm plot essentially as depicted in FIG. 4.

It is well known that the DVS isotherm plots may vary due to, for example, the purity of the sample and sample size and due to instrumental parameters, especially the equilibrium criteria settings used during the experiment. Hence, a person of skill in the art understands that the DVS data presented are not to be taken as absolute values.

In a yet still further embodiment, Form I of Compound I has a DVS isotherm plot comprising a mass gain of less than about 3% (e.g., less than about 2.5%, less than about 2%, less than about 1.5%, or less than 1%) of the total mass of the sample when increased from about 0% relative humidity (RH) to about 90% RH at about ambient temperature.

In an even still further embodiment, Form I of Compound I has a DVS isotherm plot comprising a mass gain of between about 1.2% and about 1.6% (e.g., about 1.4%) of the total mass of the sample when increased from about 0% RH to about 90% RH at about ambient temperature.

In a yet still even further embodiment, Form I of Compound I has a DVS isotherm plot comprising a mass gain of less than about 2% (e.g., less than about 1.5%, less than about 1%, or less than about 0.5%) of the total mass of the sample when increased from about 0% RH to about 70% RH at about ambient temperature.

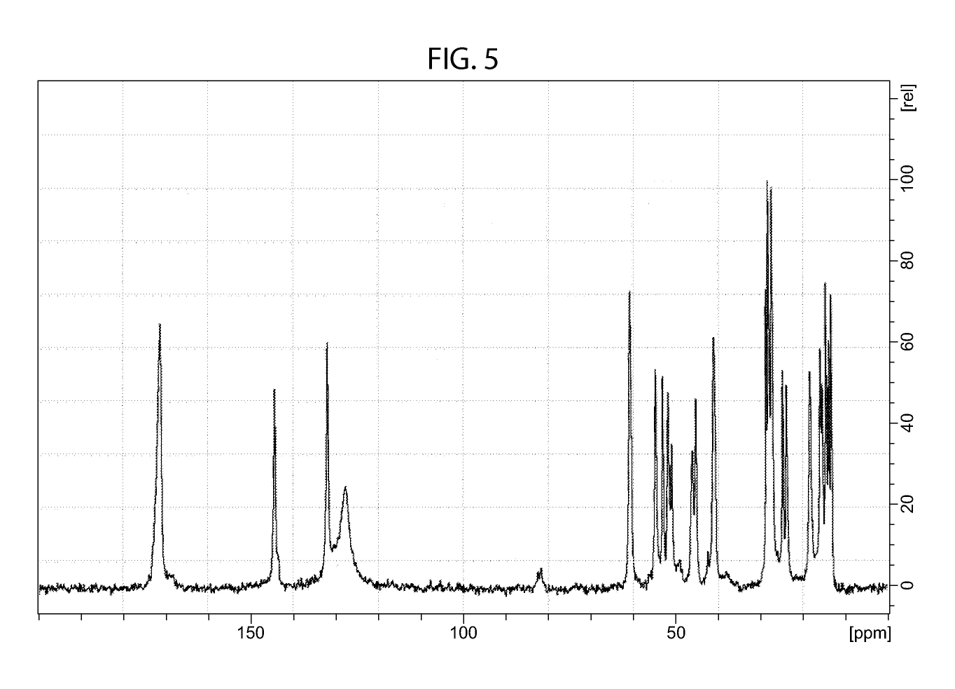

Yet another embodiment relates to Form I of Compound I that has a CP-MAS SS-NMR spectrum essentially as depicted in FIG. 5.

In a further embodiment, Form I of Compound I has a CP-MAS SS-NMR spectrum exhibiting a peak at any one, two, three, four, five, six, seven, eight, nine, ten, eleven, twelve, or more of the following ppm values: about 171.06; about 144.17; about 131.76; about 127.53; about 60.47; about 54.52; about 52.92; about is 51.56; about 50.78; about 45.95; about 45.04; about 40.79; about 28.50; about 24.58; about 23.71; about 18.13; about 15.75; about 15.29; about 14.37; about 13.67; and about 13.11 ppm.

In a still further embodiment, Form I of Compound I has a CP-MAS SS-NMR spectrum exhibiting peaks at about 171.1 ppm, about 144.2 ppm, and about 131.8 ppm.

In yet still as further embodiment, Form I of Compound I has a CP-MAS SS-NMR spectrum exhibiting peaks at about 60.5 ppm and about 40.8 ppm.

In an even further embodiment, Form I of Compound I has a CP-MAS SS-NMR spectrum exhibiting a peak at about 28.5 ppm.

In an even still further embodiment, Form I of Compound I has a CP-MAS SS-NMR spectrum exhibiting a peak at about 18.1 ppm.

In yet another embodiment, Form I of Compound I has a CP-MAS SS-NMR spectrum exhibiting peaks at about 14.4 ppm, about 13.7 ppm, and about 13.1 ppm.

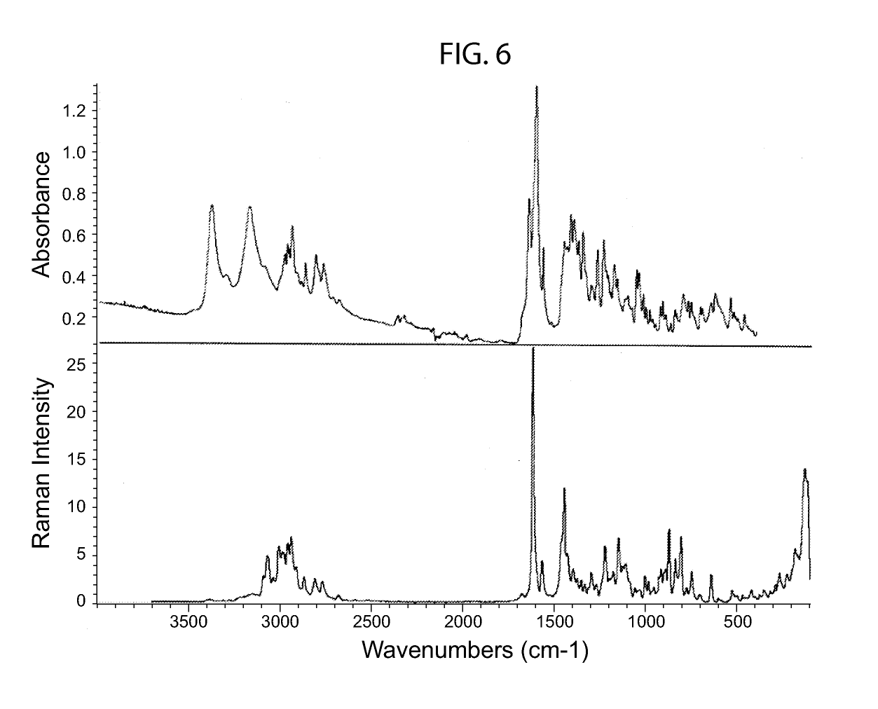

Another embodiment relates to Form I of Compound I that has an FT-IR spectrum essentially as depicted in FIG. 6 (top spectrum).

In another embodiment, Form I of Compound I has an FT-IR spectrum exhibiting a peak at about 3378.97 cm⁻¹.

In still another embodiment, Form I of Compound I has an FT-IR spectrum exhibiting a peak at about 3171.70 cm⁻¹.

In yet still another embodiment, Form I of Compound I has an FT-IR spectrum exhibiting a peak at about 2939.02 cm⁻¹.

In an even still further embodiment, Form I of Compound I has an FT-IR spectrum exhibiting a peak at about 2808.65 cm⁻¹.

In yet another embodiment, Form I of Compound I has an FT-IR spectrum exhibiting a peak at about 1646.80 cm⁻¹.

In still another embodiment, Form I of Compound I has an FT-IR spectrum exhibiting a peak at about 1607.63 cm⁻¹.

In an even further embodiment, Form I of Compound I has an FT-IR spectrum exhibiting a peak at about 1567.34 cm⁻¹.

In still a further embodiment, Form I of Compound I has an FT-IR spectrum exhibiting a peak at about 1414.45 cm⁻¹.

In yet a further embodiment, Form I of Compound I has an FT-IR spectrum exhibiting a peak at about 1234.13 cm⁻¹.

In still yet a further embodiment, Form I of Compound I has an FT-IR spectrum exhibiting a peak at about 1055.18 cm⁻¹.

In one embodiment, Form I of Compound I has an FT-IR spectrum exhibiting a peak at about 798.42 cm⁻¹.

Another embodiment relates to Form I of Compound I that has an FT-Raman spectrum essentially as depicted in FIG. 6 (bottom spectrum).

In a further embodiment, Form I of Compound I has an FT-Raman spectrum exhibiting a peak at about 3070.22 cm⁻¹.

In a still further embodiment, Form I of Compound I has an FT-Raman spectrum exhibiting a peak at about 3006.28 cm⁻¹.

In yet still a further embodiment, Form I of Compound I has an FT-Raman spectrum exhibiting a peak at about 2940.36 cm⁻¹.

In a still yet further embodiment, Form I of Compound I has an FT-Raman spectrum exhibiting a peak at about 2867.12cm⁻¹.

In yet another embodiment, Form I of Compound I has an FT-Raman spectrum exhibiting a peak at about 2808.64 cm⁻¹.

In still another embodiment, Form I of Compound I has an FT-Raman spectrum exhibiting a peak at about 2767.97 cm⁻¹.

In yet still another embodiment, Form I of Compound I has an FT-Raman spectrum exhibiting a peak at about 1614.44 cm⁻¹.

In an even further embodiment, Form I of Compound I has an FT-Raman spectrum exhibiting a peak at about 1562.48 cm⁻¹.

In yet a further embodiment, Form I of Compound I has an FT-Raman spectrum exhibiting a peak at about 1219.17 cm⁻¹.

In still yet a further embodiment, Form I of Compound I has an FT-Raman spectrum exhibiting a peak at about 1144.15 cm⁻¹.

In an even still further embodiment, Form I of Compound I has an FT-Raman spectrum exhibiting a peak at about 867.54 cm⁻¹.

In yet an even still further embodiment, Form I of Compound I has an FT-Raman spectrum exhibiting a peak at about 834 cm⁻¹.

In yet an even still further embodiment, Form I of Compound I has an FT-Raman spectrum exhibiting a peak at about 803.77 cm⁻¹

In one embodiment, a solid form provided herein has one or more advantageous properties. For example, in some embodiments, Form I of Compound I shows advantageous properties, such as, for example, a high melting point, a substantial lack of solvent (e.g., water) content, little or no weight loss on heating, and/or low hygroscopicity. In certain embodiments, such properties advantageously facilitate the manufacture, storage, formulation, and/or delivery of Compound 1.

Certain solid forms provided herein provide advantageous properties with regard to stability. The term “stability” as used herein includes chemical stability and solid-state stability.

Chemical stability includes the ability to store a solid form as an isolated material and/or as part of a formulation in which it is provided in admixture with pharmaceutically acceptable carriers, diluents or adjuvants (e.g., in an oral dosage form, such as a tablet, capsule, etc.), under normal storage conditions, with an insignificant degree of chemical degradation or decomposition.

Solid-state stability includes the ability to store a solid form as an isolated material and/or as part of a solid formulation in which it is provided in admixture with pharmaceutically acceptable carriers, diluents or adjuvants (e.g., in an oral dosage form, such as a tablet, capsule etc.), under normal storage conditions, with an insignificant degree of solid-state transformation (e.g., crystallization, recrystallization, solid-state phase transition, hydration, dehydration, solvation and/or desolvation).

Examples of “normal storage conditions” include temperatures of between −80° C. and 50° C. (e.g., between 0° C. and 40° C., or about room temperature, such as a temperature between about 15° C. and about 30° C.), pressures of between 0.1 and 2 bars (e.g., atmospheric pressure), relative humdiities (“RHs”) of between 5% and 95% (e.g., 10% to 60% RH), and/or exposure to 460 lux of UV/visible light, for prolonged periods (e.g., greater than or equal to six months). Under such conditions, solid forms provided herein may be found to be less than 15%, less than 10%, or less than 5% chemically degraded/decomposed, or solid-state transformed, as appropriate. The skilled person will appreciate that the above-mentioned upper and lower limits for temperature, pressure, and RH represent extremes of normal storage conditions, and that certain combinations of these extremes may not be experienced during normal storage (e.g., a temperature of 50° C. and a pressure of 0.1 bar).

### Processes for Preparing the Solid Forms

Further embodiments provide processes for preparing the solid forms provided herein. Alternative conditions under which the solid forms may be prepared may be determined by the skilled person using information provided herein in combination with techniques and methods known in the art. Experimental temperatures and times depend upon the solid form that is to be isolated, the concentration of the compound in solution, and the solvent system used. Crystallization may be initiated and/or effected by way of standard techniques, for example with or without seeding with crystals of the solid form.

Certain embodiments herein relate to a process for preparing Form I of Compound I. In certain embodiments, Form I is prepared by a process comprising dissolving Compound I in one or more suitable solvent(s), and isolating Form I. In certain embodiments, Form I is prepared by a process comprising slurrying Compound I in one or more suitable solvent(s), and isolating Form I. In certain embodiments, the slurrying is performed at ambient temperature. In certain embodiments, the slurrying is performed for about 3 days. In certain embodiments, the isolated Form I of Compound I is dried in air. In certain embodiments, the starting Compound I material for processes provided herein is an amorphous solid form of Compound I. In certain embodiments, a suitable solvent is selected from EtOAc or ACN or a mixture thereof.

In certain embodiments, a suitable solvent for use in a process for preparing Form I of Compound I may be selected from polar aprotic solvents (e.g., DMSO, DMF); acetates (e.g., C₁₋₆-alkyl acetates, ethyl acetate, iso-propyl acetate); alcohols (e.g., lower alkyl alcohols, linear or branched C₁₋₆-alkyl alcohols, methanol, ethanol, iso-propanol, 1-propanol); hydrocarbons (e.g., aliphatic and aromatic hydrocarbons, C₆₋₁₂-aliphatic hydrocarbons, C₆₋₁₀-aromatic hydrocarbons, n-heptane); ethers (e.g., dialkyl ethers, di-C₁₋₆-alkyl ethers, diethyl ether); ketones (e.g., dialkyl ketones, di-C₁₋₆-alkyl ketones, acetone, methyl iso-butyl ketone); nitriles (e.g., acetonitrile); chlorinated solvents (e.g., chlorinated alkanes, chlorinated methanes, chlorinated ethanes, dichloromethane); aqueous solvents (e.g., water or solvents containing water); and mixtures thereof.

It will be appreciated by the skilled person that solid forms provided herein may be prepared by analogy with processes described herein and/or in accordance with the Examples herein, and solid forms prepared according to such analogous processes may show essentially the same XRPD characteristics as disclosed herein. The term “essentially” when used as part of a comparison between data (e.g., two XRPD patterns) includes those instances when it is clear to the skilled person from the relevant data that they correspond to the same solid form, upon allowing for, e.g., experimental error and sample-to-sample variation.

### Methods of Using the Solid Forms

In one embodiment, at least one solid form comprising Compound I described herein may be used to modulate at least one histamine H3 receptor. The terms “modulate”, “modulates”, “modulating”, or “modulation”, as used herein, refer to, for example, the activation (e.g., agonist activity) or inhibition (e.g., antagonist and inverse agonist activity) of at least one histamine H3 receptor. In one embodiment, at least one solid form described herein may be used as an inverse agonist of at least one histamine H3 receptor. In another embodiment, at least one solid form described herein may be used as an antagonist of at least one histamine H3 receptor. In another embodiment, at least one solid form described herein may be used as an antagonist of at least one histamine H3 receptor. In yet another embodiment, at least one solid form described herein may be used an antagonist of at least one histamine H3 receptor.

At least one solid form described herein may be used to treat one or more of a wide range of conditions or disorders in which modulating the histamine H3 receptor is beneficial. At least one solid form described herein may, for example, be useful to treat at least one disease of the central nervous system, the peripheral nervous system, the cardiovascular system, the pulmonary system, the gastrointestinal system, or the endocrinological system.

Another embodiment provides a method for treating a disorder in which modulating the function of at least one histamine H3 receptor is beneficial comprising administering to a warm-blooded animal in need of such treatment a therapeutically effective amount of Form I of Compound I.

One embodiment relates to the use of Form I of Compound I in the manufacture of a medicament for the treatment of at least one disorder selected from schizophrenia, narcolepsy, excessive daytime sleepiness, obesity, attention deficit hyperactivity disorder, pain, Alzheimer's disease, cognition deficiency, and cognition deficiency associated with schizophrenia.

Another embodiment relates to the use of Form I of Compound I in the manufacture of a medicament for the treatment of at least one disorder selected from schizophrenia, narcolepsy, obesity, attention deficit hyperactivity disorder, pain, Alzheimer's disease, cognition deficiency, and cognition deficiency associated with schizophrenia.

A further embodiment relates to a method for the therapy of at least one disorder selected from schizophrenia, narcolepsy, excessive daytime sleepiness, obesity, attention deficit hyperactivity disorder, pain, Alzheimer's disease, cognition deficiency, and cognition deficiency associated with schizophrenia, in a warm-blooded animal in need of such therapy, wherein the method comprises administering to the animal a therapeutically effective amount of Form I of Compound I.

A still further embodiment relates to a method for the therapy of at least one disorder selected from schizophrenia, narcolepsy, obesity, attention deficit hyperactivity disorder, pain, Alzheimer's disease, cognition deficiency, and cognition deficiency associated with schizophrenia, in a warm-blooded animal in need of such therapy, wherein the method comprises administering to the animal a therapeutically effective amount of Form I of Compound I.

A further embodiment relates to a method for the treatment of at least one disorder selected from schizophrenia, narcolepsy, excessive daytime sleepiness, obesity, attention deficit hyperactivity disorder, pain, Alzheimer's disease, cognition deficiency, and cognition deficiency associated with schizophrenia, whereby a pharmaceutically and pharmacologically effective amount of Form I of Compound I is administered to a subject in need of such treatment.

Form I of Compound I may be useful to treat at least one autoimmune disorder.

Exemplary autoimmune disorders include, but are not limited to, for example, arthritis, skin grafts, organ transplants and similar surgical needs, collagen diseases, various allergies, tumors and viruses.

Form I of Compound I may be useful to treat at least one psychiatric disorder.

Exemplary psychiatric disorders include, but are not limited to, for example, Psychotic Disorder(s) and Schizophrenia Disorder(s), such as, for example, Schizoaffective Disorder(s), Delusional Disorder(s), Brief Psychotic Disorder(s), Shared Psychotic Disorder(s), and Psychotic Disorder(s) Due to a General Medical Condition; Dementia and other Cognitive Disorder(s); Anxiety Disorder(s), such as, for example, Panic Disorder(s) Without Agoraphobia, Panic Disorder(s) With Agoraphobia, Agoraphobia Without History of Panic Disorder(s), Specific Phobia, Social Phobia, Obsessive-Compulsive Disorder(s), Stress related Disorder(s), Posttraumatic Stress Disorder(s), Acute Stress Disorder(s), Generalized Anxiety Disorder(s) and Generalized Anxiety Disorder(s) Due to a General Medical Condition; Mood Disorder(s), such as, for example, a) Depressive Disorder(s) (including but not limited to, for example, Major Depressive Disorder(s) including depression, major depression, mood stabilization and/or apathy, and Dysthymic Disorder(s)), b) Bipolar Depression and/or Bipolar mania, such as, for example, Bipolar I (which includes, but is not limited to those with manic, depressive or mixed episodes), Bipolar II, and Bipolar Maintenance, c) Cyclothymiac's Disorder(s), and d) Mood Disorder(s) Due to a General Medical Condition; Sleep Disorder(s), such as, for example, excessive daytime sleepiness, narcolepsy, hypersomina, and sleep apnea; Disorder(s) Usually First Diagnosed in Infancy, Childhood, or Adolescence including, but not limited to, for example, Mental Retardation, Downs Syndrome, Learning Disorder(s), Motor Skills Disorder(s), Communication Disorders(s), Pervasive Developmental Disorder(s), Attention-Deficit and Disruptive Behavior Disorder(s), Feeding and Eating Disorder(s) of Infancy or Early Childhood, Tic Disorder(s), and Elimination Disorder(s); Substance-Related Disorder(s) including, but not limited to, for example, Substance Dependence, Substance Abuse, Substance Intoxication, Substance Withdrawal, Alcohol-Related Disorder(s), Amphetamines (or Amphetamine-Like)-Related Disorder(s), Caffeine-Related Disorder(s), Cannabis-Related Disorder(s), Cocaine-Related Disorder(s), Hallucinogen-Related Disorder(s), Inhalant-Related Disorder(s), Nicotine-Related Disorder(s), Opiod-Related Disorder(s), Phencyclidine (or Phencyclidine-Like)-Related Disorder(s), and Sedative-, Hypnotic- or Anxiolytic-Related Disorder(s); Attention-Deficit and Disruptive Behavior Disorder(s); Eating Disorder(s), such as, for example, obesity; Personality Disorder(s) including, but not limited to, for example, Obsessive-Compulsive Personality Disorder(s); Impulse-Control Disorder(s); Tic Disorders including, but not limited to, for example Tourette's Disorder, Chronic motor or vocal tic disorder; and Transient Tic Disorder. At least one of the above psychiatric disorders is defined, for example, in the American Psychiatric Association: Diagnostic and Statistical Manual of Mental Disorders, Fourth Edition, Text Revision, Washington, D.C., American Psychiatric Association, 2000.

Form I of Compound I may be useful: i) to treat obesity or being overweight (e.g., promotion of weight loss and maintenance of weight loss), eating disorders (e.g., binge eating, anorexia, bulimia and compulsive), and/or cravings (for drugs, tobacco, alcohol, any appetizing macronutrients or non-essential food items); ii) to prevent weight gain (e.g., medication-induced or subsequent to cessation of smoking); and/or iii) to modulate appetite and/or satiety. At least one solid form described herein may be suitable for treating obesity by reducing appetite and body weight and/or maintaining weight reduction and preventing rebound. At least one solid form described herein may be used to prevent or reverse medication-induced weight gain, e.g., weight gain caused by antipsychotic (neuroleptic) treatment(s); and/or weight gain associated with smoking cessation.

Form I of Compound I may be useful to treat at least one Neurodegenerative Disorder.

Exemplary Neurodegenerative Disorders include, but are not limited to, for example, conditions associated with cognitive disorder(s) or indications with deficit(s) in cognition such as: dementia; incl. pre-senile dementia (early onset Alzheimer's Disease); senile dementia (dementia of the Alzheimer's type); Alzheimer's Disease (AD); Familial Alzheimer's disease; Early Alzheimer's disease; mild to moderate dementia of the Alzheimer's type; delay of disease progression of Alzheimer's Disease; neurodegeneration associated with Alzheimer's disease, Mild Cognitive Impairment (MCI); Amnestic Mild Cognitive Impairment (aMCI); Age-associated Memory Impairment (AAMI); Lewy body dementia; vascular dementia (VD); HIV-dementia; AIDS dementia complex; AIDS-Neurological Complications; Frontotemporal dementia (FTD); Frontotemporal dementia Parkinson's Type (FTDP); dementia pugilistica; dementia due to infectious agents or metabolic disturbances; dementia of degenerative origin; dementia-Multi-Infarct; memory loss; cognition in Parkinson's Disease; cognition in multiple sclerosis; cognition deficits associated with chemotherapy; Cognitive Deficit in Schizophrenia (CDS); Schizoaffective disorders including schizophrenia; Age-Related Cognitive Decline (ARCD); Cognitive Impairment No Dementia (CIND); Cognitive Deficit arising from stroke or brain ischemia; Congenital and/or development disorders; progressive supranuclear palsy (PSP); amyotrophic lateral sclerosis (ALS); corticobasal degeneration(CBD); traumatic brain injury (TBI); postencephelatic parkinsonism; Pick's Disease; Niemann-Pick's Disease; Down's syndrome; Huntington's Disease; Creurtfeld-Jacob's disease; prion diseases; multiple sclerosis (MS); motor neuron diseases (MND); Parkinson's Disease (PD); β-amyloid angiopathy; cerebral amyloid angiopathy; Trinucleotide Repeat Disorders; Spinal Muscular Atrophy; Ataxia; Friedreich's Ataxia; Ataxias and Cerebellar or Spinocerebellar Degerneration; Neuromyelitis Optica; Multiple System Atrophy; Transmissible Spongiform Encephalopathies; Attention Deficit Disorder (ADD); Attention Deficit Hyperactivity Disorder (ADHD); Bipolar Disorder (BD) including acute mania, bipolar depression, bipolar maintenance; Major Depressive Disorders (MDD) including depression, major depression, mood disorder (stabilization), dysthymia and apathy; Guillain-Barré Syndrome (GBS); and Chronic Inflammatory Demyelinating Polyneuropathy (CIDP).

Form I of Compound I may be useful to treat at least one Neuroinflammatory Disorder including, but not limited to, for example, Multiple Sclerosis (MS), which includes, but is not limited to, for example, Relapse Remitting Multiple Sclerosis (RRMS), Secondary Progressive Multiple Sclerosis (SPMS), and Primary Progressive Multiple Sclerosis (PPMS); Parkinson's disease; Multiple System Atrophy (MSA); Corticobasal Degeneration; Progressive Supranuclear Paresis; Guillain-Barré Syndrome (GBS); and chronic inflammatory demyelinating polyneuropathy (CIDP).

Form I of Compound I may be useful to treat at least one Attention-Deficit and Disruptive Behavior Disorder.

Exemplary Attention-Deficit and Disruptive Behavior Disorders include, but are not limited to, for example, attention deficit disorder (ADD), attention deficit hyperactivity disorder (ADHD), and affective disorders.

Form I of Compound I may be useful to treat pain, including acute or chronic pain disorders including but not limited to, for example, Widespread pain, Localized pain, Nociceptive pain, Inflammatory pain, Central pain, Central and peripheral neuropathic pain, Diabetic neuropathic pain, Central and peripheral neurogenic pain, Central and peripheral neuralgia, Low back pain, Postoperative pain, Visceral pain, and Pelvic pain; Allodynia; Anesthesia dolorosa; Causalgia; Dysesthesia; Fibromyalgia; Hyperalgesia; Hyperesthesia; Hyperpathia; Ischemic pain; Sciatic pain; Burn-induced pain; Pain associated with cystitis including, but not limited to, interstitial cystitis; Pain associated with multiple sclerosis; Pain associated with arthritis; Pain associated with osteoarthritis; Pain associated with rheumatoid arthritis; Pain associated with pancreatitis; Pain associated with psoriasis; Pain is associated with fibromyalgia; Pain associated with IBS; Pain associated with cancer; and Restless Legs Syndrome.

Form I of Compound I may be useful to treat at least one of the following disorders Autism, Dyslexia, Jetlag, Hyperkinesias, Dystonias, Rage outbursts, Muscular Dystrophy, Neurofibromatosis, Spinal Cord Injury, Cerebral Palsy, Neurological Sequelae of Lupus and Post-Polio Syndrome.

Form I of Compound I may be used for the manufacture of a medicament for the treatment of at least one autoimmune disorder, psychiatric disorder, obesity disorder, eating disorder, craving disorder, neurodegenerative disorder, neuroinflammatory disorder, Attention-Deficit and Disruptive Behaviour Disorder, and/or pain disorder described hereinabove.

Form I of Compound I may be used for the manufacture of a medicament for the treatment of at least one disorder selected from cognitive deficit in schizophrenia, narcolepsy, excessive daytime sleepiness, attention deficit hyperactivity disorder, obesity, pain, and Alzheimer's disease.

Form I of Compound I may be used for the manufacture of a medicament for the treatment of at least one disorder selected from cognitive deficit in schizophrenia, narcolepsy, attention deficit hyperactivity disorder, obesity, pain, and Alzheimer's disease.

Form I of Compound I may be used for the treatment of at least one disorder selected from cognitive deficits in schizophrenia, narcolepsy, excessive daytime sleepiness, obesity, attention deficit hyperactivity disorder, pain, and Alzheimer's disease. Form I of Compound I may be used for the treatment of at least one disorder selected from cognitive deficits in schizophrenia, narcolepsy, obesity, attention deficit hyperactivity disorder, pain, and Alzheimer's disease.

Form I of Compound I may be used for the treatment of at least one disorder selected from cognitive deficits in schizophrenia and Alzheimer's disease. Another aspect provides a method for treating at least one autoimmune disorder, psychiatric disorder, obesity disorder, eating disorder, craving disorder, neurodegenerative disorder, neuroinflammatory disorder, attention-deficit and disruptive behaviour disorder, and/or pain disorder in a warm-blooded animal, comprising administering to said animal in need of such treatment a therapeutically effective amount of Form I of Compound I.

Yet another aspect provides a method for treating at least one disorder selected from cognitive deficits in schizophrenia, narcolepsy, excessive daytime sleepiness, obesity, attention deficit hyperactivity disorder, pain, and Alzheimer's disease in a warm-blooded animal, comprising administering to said animal in need of such treatment a therapeutically effective amount of Form I of Compound I.

Yet another aspect provides a method for treating at least one disorder selected from cognitive deficits in schizophrenia, narcolepsy, obesity, attention deficit hyperactivity disorder, pain, and Alzheimer's disease in a warm-blooded animal, comprising administering to said animal in need of such treatment a therapeutically effective amount of Form I of Compound I.

Yet another aspect provides a method for treating cognitive deficits in schizophrenia in a warm-blooded animal, comprising administering to said animal in need of such treatment a therapeutically effective amount of Form I of Compound I.

Yet another aspect provides a method for treating obesity in a warm-blooded animal, comprising administering to said animal in need of such treatment a therapeutically effective amount of Form I of Compound I.

Yet another aspect provides a method for treating narcolepsy in a warm-blooded animal, comprising administering to said animal in need of such treatment a therapeutically effective amount of Form I of Compound I.

Yet another aspect provides a method for treating excessive daytime sleepiness in a warm-blooded animal, comprising administering to said animal in need of such treatment a therapeutically effective amount of Form I of Compound I.

Still another aspect provides a method for treating Alzheimer's disease in a warm-blooded animal, comprising administering to said animal in need of such treatment a therapeutically effective amount of Form I of Compound I.

Still yet another aspect provides a method for treating attention deficit hyperactivity disorder in a warm-blooded animal, comprising administering to said animal in need of such treatment a therapeutically effective amount of Form I of Compound I.

Yet still another aspect provides a method for treating a pain disorder in a warm-blooded animal, comprising administering to said animal in need of such treatment a therapeutically effective amount of Form I of Compound I.

In one embodiment, the warm-blooded animal is a mammalian species including, but not limited to, for example, humans and domestic animals, such as, for example, dogs, cats, and horses. In one embodiment, the warm-blooded animal is a human.

Another aspect provides the use of Form I of Compound I in therapy. Another embodiment provides the use of Form I of Compound I in the manufacture of a medicament for use in therapy. As used herein, the term “therapy” also includes “prophylaxis” unless specifically indicated to the contrary.

In yet another embodiment, Form I of Compound I, or a pharmaceutical composition or formulation comprising Form I of Compound I, may be administered concurrently, simultaneously, sequentially or separately with at least one other pharmaceutically active compound selected from the following:

(i) antidepressants including for example agomelatine, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, ramelteon, reboxetine, robalzotan, sertraline, sibutramine, thionisoxetine, tranylcypromaine, trazodone, trimipramine, venlafaxine and equivalents and pharmaceutically active isomer(s) and metabolite(s) thereof;

(ii) atypical antipsychotics including for example quetiapine and pharmaceutically active isomer(s) and metabolite(s) thereof;

(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof;

(iv) anxiolytics including for example alnespirone, azapirones,benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof;

(v) anticonvulsants including for example carbamazepine, clonazepam, ethosuximide, felbamate, fosphenytoin, gabapentin, lacosamide, lamotrogine, levetiracetam, oxcarbazepine, phenobarbital, phenytoin, pregabaline, rufinamide, topiramate, valproate, vigabatrine, zonisamide, and equivalents and pharmaceutically active isomer(s) and metabolite(s) thereof;

(vi) Alzheimer's therapies including for example donepezil, rivastigmine, galantamine, memantine, and equivalents and pharmaceutically active isomer(s) and metabolite(s) thereof;

(vii) Parkinson's therapies including for example levodopa, dopamine agonists such as apomorphine, bromocriptine, cabergoline, pramipexol, ropinirole, and rotigotine, MAO-B inhibitors such as selegeline and rasagiline, and other dopaminergics such as tolcapone and entacapone, A-2 inhibitors, dopamine reuptake inhibitors, NMDA antagonists, Nicotine agonists, and inhibitors of neuronal nitric oxide synthase and equivalents and pharmaceutically active isomer(s) and metabolite(s) thereof;

(viii) migraine therapies including for example almotriptan, amantadine, bromocriptine, butalbital, cabergoline, dichloralphenazone, dihydroergotamine, eletriptan, frovatriptan, lisuride, naratriptan, pergolide, pizotiphen, pramipexole, rizatriptan, ropinirole, sumatriptan, zolmitriptan, zomitriptan, and equivalents and pharmaceutically active isomer(s) and metabolite(s) thereof;

(ix) stroke therapies including for example thrombolytic therapy with eg activase and desmoteplase, abciximab, citicoline, clopidogrel, eptifibatide, minocycline, and equivalents and pharmaceutically active isomer(s) and metabolite(s) thereof;

(x) urinary incontinence therapies including for example darafenacin, falvoxate, oxybutynin, propiverine, robalzotan, solifenacin, tolterodine and and equivalents and pharmaceutically active isomer(s) and metabolite(s) thereof;

(xi) neuropathic pain therapies including lidocain, capsaicin, and anticonvulsants such as gabapentin, pregabalin, and antidepressants such as duloxetine, venlafaxine, amitriptyline, klomipramine, and equivalents and pharmaceutically active isomer(s) and metabolite(s) thereof;

(xii) nociceptive pain therapies including paracetamol, NSAIDS and coxibs, such as celecoxib, etoricoxib, lumiracoxib, valdecoxib, parecoxib, diclofenac, loxoprofen, naproxen, ketoprofen, ibuprofen, nabumeton, meloxicam, piroxicam and opioids such as morphine, oxycodone, buprenorfin, tramadol and equivalents and pharmaceutically active isomer(s) and metabolite(s) thereof;

(xiii) insomnia therapies including for example agomelatine, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, ramelteon, roletamide, triclofos,secobarbital, zaleplon, zolpidem and equivalents and pharmaceutically active isomer(s) and metabolite(s) thereof;

(xiv) mood stabilizers including for example carbamazepine, divalproex, gabapentin, lamotrigine, lithium, olanzapine, quetiapine, valproate, valproic acid, verapamil, and equivalents and pharmaceutically active isomer(s) and metabolite(s) thereof;

(xv) obesity therapies, such as, for example, anti-obesity drugs that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, and G-I motility; very low calorie diets (VLCD); and low-calorie diets (LCD);

(xvi) therapeutic agents useful in treating obesity associated disorders, such as, for example, biguanide drugs, insulin (synthetic insulin analogues) and oral antihyperglycemics (these are divided into prandial glucose regulators and alpha-glucosidase inhibitors), PPAR modulating agents, such as, for example, PPAR alpha and/or gamma agonists; sulfonylureas; cholesterol-lowering agents, such as, for example, inhibitors of HMG-CoA reductase (3-hydroxy-3-methylglutaryl coenzyme A reductase); an inhibitor of the ileal bile acid transport system (IBAT inhibitor); a bile acid binding resin; bile acid sequestering agent, such as, for example, colestipol, cholestyramine, or cholestagel; a CETP (cholesteryl ester transfer protein) inhibitor; a cholesterol absorption antagonist; a MTP (microsomal transfer protein) inhibitor; a nicotinic acid derivative, including slow release and combination products; a phytosterol compound; probucol; an anti-coagulant; an omega-3 fatty acid; an anti-obesity therapy, such as, for example, sibutramine, phentermine, orlistat, bupropion, ephedrine, and thyroxine; an antihypertensive, such as, for example, an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic, and a vasodilator; a melanin concentrating hormone (MCH) modulator; an NPY receptor modulator; an orexin receptor modulator; a phosphoinositide-dependent protein kinase (PDK) modulator; modulators of nuclear receptors, such as, for example, LXR, FXR, RXR, GR, ERRα, β, PPARα, β, γ and RORalpha; a monoamine transmission-modulating agent, such as, for example, a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA); a serotonin receptor modulator; a leptin/leptin receptor modulator; a ghrelin/ghrelin receptor modulator; a DPP-IV inhibitor; and equivalents and pharmaceutically active isomer(s), metabolite(s), and pharamaceutically acceptable salts, solvates, and prodrugs thereof;

(xvii) agents for treating ADHD, such as, for example, amphetamine, methamphetamine, dextroamphetamine, atomoxetine, methylphenidate, dexmethylphenidate, modafinil, and equivalents and pharmaceutically active isomer(s) and metabolite(s) thereof; and

(xviii) agents used to treat substance abuse disorders, dependence, and withdrawal, such as, for example, nicotine replacement therapies (i.e., gum, patches, and nasal spray); nicotinergic receptor agonists, partial agonists, and antagonists, (e.g., varenicline); acomprosate, bupropion, clonidine, disulfiram, methadone, naloxone, naltrexone, and equivalents and pharmaceutically active isomer(s) and metabolite(s) thereof.

When employed in combination with at least one solid form described herein, the above other pharmaceutically active compound may be used, for example, in the amounts indicated in the Physicians' Desk Reference (PDR; e.g., 64th ed. 2010) or approved dosage ranges and/or dosage described in published references or as otherwise determined by one of ordinary skill in the art.

Solid forms described herein may be administered by any means suitable for the condition to be treated, which can depend on the quantity of the solid form described herein to be delivered. Solid form(s) described herein may be administered in the form of a pharmaceutical composition by any route including, but not limited to, for example, orally, intramuscularly, subcutaneously, topically, intranasally, epidurally, intraperitoneally, intrathoracially, intravenously, intrathecally, intracerebroventricularly, and injecting into the joints. In one embodiment, the route of administration is orally.

An “effective amount” of a solid form described herein may be determined by one of ordinary skill in the art. For example, the quantity of the solid form to be administered will vary for the patient being treated, and may vary from about 100 ng/kg of body weight to 100 mg/kg of body weight per day (e.g., from 10 pg/kg to 10 mg/kg per day). In particular embodiments, an effective amount includes exemplary dosage amounts for a mammal of from about 0.05 to about 300 mg/kg/day (e.g., less than about 200 mg/kg/day) in a single dose or in or in the form of individual divided doses. In particular embodiments, exemplary dosage amounts for an adult human are from about 1 to 100 mg/kg of body weight per day (e.g., 15 mg/kg of body weight per day), which can be administered in a single dose or in the form of individual divided doses, such as from 1 to 4 times per day.

Dosages can be readily ascertained by those skilled in the art based on this disclosure and the knowledge in the art. Thus, the skilled person can readily determine the amount of solid form and optional additives, vehicles, and/or carriers in compositions and to be administered in methods provided herein. The specific dose level and frequency of dosage for any particular subject, however, may vary and generally depends on a variety of factors, including, but not limited to, for example, the dissolution and/or bioavailability of the solid form(s) described herein; species, age, body weight, general health, sex, and diet of the subject; mode and time of administration; rate of excretion; drug combination; and severity of the particular condition.

### Pharmaceutical Compositions Comprising the Solid Forms

One aspect provides a pharmaceutical composition comprising Form I of Compound I and at least one pharmaceutically-acceptable carrier and/or diluent.

One embodiment provides a method for treating at least one disorder described herein in a warm-blooded animal, comprising administering to said animal in need of such treatment a pharmaceutical composition comprising a therapeutically effective amount of Form I of Compound I, and at least one pharmaceutically-acceptable carrier and/or diluent.

One embodiment provides a method for treating at least one disorder selected from cognitive deficits in schizophrenia, narcolepsy, excessive daytime sleepiness, obesity, attention deficit hyperactivity disorder, and Alzheimer's disease in a warm-blooded animal, comprising administering to said animal in need of such treatment a pharmaceutical composition comprising a therapeutically effective amount of Form I of Compound I, and at least one pharmaceutically-acceptable carrier and/or diluent.

One embodiment provides a method for treating at least one disorder selected from cognitive deficits in schizophrenia, narcolepsy, obesity, attention deficit hyperactivity disorder, and Alzheimer's disease in a warm-blooded animal, comprising administering to said animal in need of such treatment a pharmaceutical composition comprising a therapeutically effective amount of Form I of Compound I, and at least one pharmaceutically-acceptable carrier and/or diluent.

Acceptable solid pharmaceutical compositions include, but are not limited to, for example, powders, tablets, dispersible granules, capsules, cachets, and suppositories. In a solid pharmaceutical composition, pharmaceutically acceptable carriers include, but are not limited to, for example, at least one solid, at least one liquid, and mixtures thereof. The solid carrier can also be a diluent, flavoring agent, solubilizer, lubricant, suspending agent, binder, encapsulating material, and/or tablet-disintegrating agent. Suitable carriers, include, but are not limited to, for example, magnesium carbonate; magnesium stearate; talc; lactose; sugar; pectin; dextrin; starch; tragacanth; methyl cellulose; sodium carboxymethyl cellulose; a low-melting wax; cocoa butter; and mixtures thereof. Examples of suitable carriers are known to the skilled person and are described, e.g., in Remington: The Science and Practice of Pharmacy (Lippincott Williams & Wilkins, 20th ed. 2000).

A powder can be prepared by, for example, mixing a finely divided solid with Form I of Compound I. A tablet can be prepared by, for example, mixing Form I of Compound I in suitable proportions with a pharmaceutically acceptable carrier having the necessary binding properties and compacted into the desired shape and size. A suppository can be prepared by, for example, mixing Form I of Compound I with at least one suitable non-irritating excipient that is liquid at rectal temperature but solid at a temperature below rectal temperature, wherein the non-irritating is excipient is first melted and Form I of Compound I is dispersed therein. The molten homogeneous mixture is then poured into convenient sized molds and allowed to cool and solidify. Exemplary non-irritating excipients include, but are not limited to, for example, cocoa butter; glycerinated gelatin; hydrogenated vegetable oils; mixtures of polyethylene glycols of various molecular weights; and fatty acid esters of polyethylene glycol.

Acceptable liquid pharmaceutical compositions include suspensions. Aqueous suspensions for oral administration can be prepared by dispersing at least one finely divided solid form described herein in water together with a viscous material, such as, for example, a natural synthetic gum, resin, methyl cellulose, and sodium carboxymethyl cellulose.

In one embodiment, a pharmaceutical composition described herein contains between about 0.05% and about 99% (by weight) of Form I of Compound I (all percentages by weight being based on total composition). In another embodiment, a pharmaceutical composition contains from about 0.10% to about 50% (by weight) of Form I of Compound I (all percentages by weight being based on total composition).

Another embodiment provides a pharmaceutical composition comprising Form I of Compound I, and a pharmaceutically acceptable carrier/diluent for therapy.

Further, there is provided a pharmaceutical composition comprising Form I of Compound I, in association with a pharmaceutically acceptable carrier for use in any of the conditions discussed hereinabove.

### EXAMPLES

The invention is further defined in the following Examples. It should be understood that the Examples are given by way of illustration only. From the above discussion and the Examples, one skilled in the art can ascertain the essential characteristics of the invention, and without departing from the spirit and scope thereof, can make various changes and modifications to adapt the invention to various uses and conditions. As a result, the invention is not limited by the illustrative examples set forth hereinbelow, but rather defined by the claims appended hereto.

All temperatures are in degrees Celsius (° C.) and are uncorrected.

Unless otherwise noted, commercial reagents used in preparing the example compounds were used as received without additional purification.

Unless otherwise noted, the solvents used in preparing the example compounds were commercial anhydrous grades and were used without further drying or purification.

All starting materials are commercially available, unless stated otherwise.

The following abbreviations may be employed herein: ACN: acetonitrile; aq: aqueous; br: broad; Bu: butyl; calcd: calculated; Celite®: brand of diatomaceous earth filtering agent, registered trader of Celite Corporation; CP-MAS SS-NMR: cross-polarization magic angle spinning solid-state nuclear magnetic resonance; d: doublet; dd: doublet of doublet; ddd: doublet of doublet of doublet; dddd: doublet of doublet of doublet of doublet; DABCO: 1,4-diazabicyclo[2.2.2]octane; DCE: dichloroethane; DCM: dichloromethane; DIPEA: N-ethyl-N-isopropylpropan-2-amine; DME: dimethyl ether; DMEA: dimethyl ethylamine; DMF: N,N-dimethyl formamide; DMSO: dimethyl sulfoxide; dq: doublet of quartet; DSC: differential scanning calorimetry; dt: doublet of triplet; DVS: dynamic vapour sorption; EDC: 1-ethyl-3-(3-dimethylaminopropyl)carbodiimide hydrochloride; ESI: electrospray ion source; EtOAc: ethyl acetate; EtOH: ethanol; Et: ethyl; FT-IR: Fourier-transform infrared; FT-Raman: Fourier transform Raman; g: gram; h: hour(s); ¹H NMR: proton nuclear magnetic resonance; HBTU: O-Benzotriazole-N,N,N′,N′-tetramethyl-uronium-hexafluoro-phosphate; HCl: hydrochloric acid; HOBT: N-Hydroxybenzotriazole; HPLC: high pressure liquid chromatography; HRMS: high resolution mass spectrometry; iPrOH: iso-propanol; L: liter; m: multiplet; M: molar; mL: milliliter; Me: methyl; MeOH: methanol; mg: milligram; MgSO₄: anhydrous magnesium sulfate (drying agent); MHz: megahertz; min: minute(s); mmol: millimole; mol: mole; MPLC: medium pressure liquid chromatography; MS: mass spectrometry; MTBE: methyl tert-butyl ether; NaHCO₃: sodium bicarbonate; NH₄Cl: ammonium chloride; Pd/C: palladium on carbon; ppm: parts per million; q: quartet; quin: quintet; rt: room temperature; s: singlet; sat: saturated; t: triplet; TEA: triethylamine; tBuOH: tert-butanol; td: triplet of doublet; TFA: trifluoroacetic acid; TGA=thermalgravimetric analysis; THF: tetrahydrofuran; UV=ultraviolet; XRPD=X-ray powder diffraction; and the prefixes n-, s-, i-, t- and tert- have their usual meanings: normal, secondary, iso, and tertiary.

### Example 1

### Synthesis of Compound I (First Route)

### 4-((trans)-2-((R)-4-cyclobutyl-2-methylpiperazine-1-carbonyl)cyclopropyl)benzamide, isomer 1.

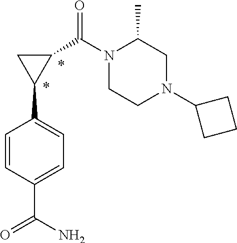

Note: * designates single isomer of unknown absolute stereochemistry.

Example 2 (138 mg, 0.40 mmol) was separated on a MettlerToledo Minigram Supercritical Fluid Chromatography instrument using the following conditions: ChiralPak AD-H, 10×250 mm, 5 μm particle size, 10.0 mL/min, mobile phase: 55% iPrOH with 0.1% DMEA, supercritical CO₂, regulator set to 100 Bar, column temperature set to 35° C., UV 215 nm, providing 57.8 mg isomer 1 (41.9%) and 56.5 mg isomer 2 (41.0%) as solids. The product was analyzed on chiral SFC (UV detection) using isocratic method (mobile phase: 55% EtOH with 0.1% DMEA, supercritical CO₂) on ChiralPak AD-H, 10×250 mm, 5 μm particle size, giving an enantiomeric purity of 99%, Rₜ 1.92 min (isomer 1) & 3.46 min (isomer 2). Isomer 1: ¹H NMR (400 MHz, CD₃OD) δ ppm 1.26 (br. s., 1 H) 1.38 (br. s., 3H) 1.59 (ddd, J=9.57, 4.69, 4.49 Hz, 1H) 1.65-1.77 (m, 3H) 1.77-1.98 (m, 3H) 1.98-2.09 (m, 2-2.31 (m, 1H) 2.43 (br. s., 1H) 2.63-2.74 (m, 2H) 2.84 (d, J=11.33 Hz, 1H) 2.96 (t, J=12.89 Hz, 0.5H), 3.36 (t, J=12.30 Hz, 0.5H) 4.04 (d, J=12.11 Hz, 0.5H) 4.31 (d, J=12.11 Hz, 0.5H) 4.38 (br. s., 0.5H) 4.65 (br. s., 0.5H) 7.25 (d, J=8.20 Hz, 2H), 7.80 (d, J=8.20 Hz, 2H); HRMS m/z calcd for C₂₀H₂₈N₃O₂ 342.21760 [M+H]⁺, found 342.21771; [α]_{D}+156.3° (c 2.20, MeOH).

### EXAMPLE 2

### 4-(trans-2-((R)-4-cyclobutyl-2-methylpiperazine-1-carbonyl)cyclopropyl)benzamide, diastereomeric mixture

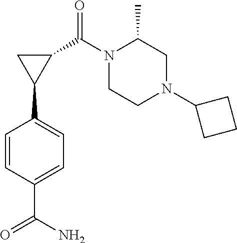

Intermediate A was dissolved in DCE (13.0 mL). TEA (0.958 mL, 6.87 mmol) was added, followed by cyclobutanone (193 mg, 2.75 mmol) and sodium triacetoxyborohydride (437 mg, 2.06 mmol). The reaction mixture was stirred is overnight and washed with sat. NaHCO₃. The organic layer was dried over MgSO₄, filtered and concentrated under reduced pressure. The crude material was purified on preparative HPLC MS using the short high pH shallow gradient method (Mobile phase: 20-40% B; A: H₂O with 10 mM NH₄CO₃ and 0.375% NH₄OH v/v, B: CH₃CN, 10 min run) on XBridge Prep C18 OBD, 30×50 mm, 5 μm, Waters reverse phase column, providing 159 mg Example 2 (33.9%) as a solid (diastereomeric mixture). ¹H NMR (400 MHz, CD₃OD) δ ppm 1.27 (d, J=7.03 Hz, 2H) 1.39 (br. s., 2H) 1.59 (ddd, J=9.18, 5.27, 4.30 Hz, 1H) 1.65-1.78 (m, 3H) 1.78-1.98 (m, 3H) 1.98-2.10 (m, 2H) 2.20-2.34 (m, 1H) 2.42 (br. s., 1H) 2.62-2.77 (m, 2H) 2.78-2.90 (m, 1H) 2.90-3.05 (m, 1H) 3.94-4.10 (m, 1H) 4.23-4.35 (m, 1H) 7.25 (d, J=8.59 Hz, 2H) 7.80 (d, J=8.20 Hz, 2H); HRMS m/z calcd for C₂₀H₂₈N₃O₂ 342.21760 [M+H]⁺, found 342.21804.

### EXAMPLE 3

### Synthesis of Compound I (Second Route)

### 4-{(1S, 2S)-2-[((R)-4-Cyclobutyl-2-methylpiperazin-1-yl)carbonyl]-cyclopropyl}-benzamide

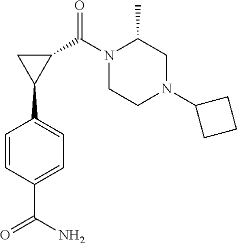

Intermediate N (10.0 g, 48.7 mmoles) was mixed in 2-MeTHF (200 mL) at t_{jacket}=25° C. 1,1′-Carbonyldiimidazole (11.0 g, 53.6 mmoles, 82.1% w/w) was added in 1 portion. The reaction slurry was slowly heated to t_{jacket}=85° C. and after approximately 5 h the reaction slurry was cooled to t_{reaction mixture}=25° C. Intermediate O (13.8 g, 58.5 mmoles) and TEA (7.55 mL, 53.6 mmoles) were added to the reaction slurry. The reaction slurry was heated at t_{jacket}=70° C. for 3 h. Analysis of a sample on HPLC indicated full conversion at this point using the gradient method (mobile phase 20-95% B; A: 5% CH₃CN in H₂O with 0.1% TFA, B: 95% CH₃CN in H₂O with 0.085% TFA, 10 min run) on Chromolith Performance RP-18e, 4.6×100 mm. The reaction slurry was cooled to t_{jacket}=40° C. 1M Na₂CO₃ in brine (90 mL) was added. The aq. phase was separated off and the organic phase was washed with brine (2 L). The assay of the title compound in the organic phase was determined by ¹H NMR and the volume of the organic phase was adjusted to 10 is relative volumes (15.4 g of title compound). The organic phase was cooled to t_{jacket}=15° C. and extracted with 10% H₃PO₄ in H₂O (charged until pH 2.5, 110 mL). The lower aq. phase was collected and the remaining organic phase was re-extracted with10% H₃PO₄ in H₂O (50 mL). The combined aq. phases were basified to pH>12 with 5M KOH and extracted with MeTHF twice (200 mL, 50 mL). The combined organic phases were extracted with brine (50 mL) and filtered to remove inorganic salts. The assay of the title compound in the organic phase was determined by ¹H NMR and the volume of the organic phase was reduced to 6 relative volumes (14.4 g of title compound, 86 mL). Crystallisation was performed starting at t_{jacket}=55° C. After cooling to t_{jacket}=40° C., heptane (21.6 mL) as well as seed (128 mg of title compound) was added. The mixture was after aging cooled down to t_{jacket}=20° C., when a second addition of heptane (64.8 mL) was performed. The product was filtered off and washed with MeTHF/Heptane twice (2*30 mL). Drying under vacuum at 40° C. gave 12.6 g title compound (35.2 mmoles, 98.7% w/w, 75% yield). ¹H-NMR (DMSO-d₆): δ 7.91 (br s, 1H), 7.78 (d, J=8.4 Hz, 2H), 7.30 (br s, 1H), 7.25 (d, J=8.0 Hz, 2H), 4.54 & 4.36 (br s, 1H), 4.17 and 4.01 (d, J=12.2 Hz, 1H), 3.20 and 2.80 (t, J=11.9 Hz, 1H), 2.74 (d, J=11.4 Hz, 1H), 2.67-2.55 (m, 2H), 2.33 (br s, 2H), 1.99-1.88 (m, 2H), 1.88-1.53 (m, 6H), 1.48-1.37 (m, 1H), 1.27 (br s, 3H), 1.12 (br s, 1H); LC-MS (ESI): m/z 342 (M+1). Rₜ 1.68 min with the analytical method (mobile phase: 5-90% B; A: H₂O with 0.1% formic acid, B: CH₃CN, 8.6 min run) on Xbridge C18, 3.0×50 mm, 2.5 μm particle size. The LC purity of the product was analyzed on an Atlantis T3 column (3.0×150 mm, 3.0 μm particle size) with UV-detection (250 nm) using a gradient method (mobile phase 2-50% B; A: H₂O with 0.03% TFA, B: CH₃CN with 0.03% TFA, 30 min run), giving a purity of 99.48 area% at 12.06 min. The product was analyzed on chiral SFC (UV detection) using isocratic method (mobile phase: 55% EtOH with 0.1% DMEA, supercritical CO₂) on ChiralPak AD-H, 10×250 mm, 5 μm particle size, giving an enantiomeric purity of >99% ee, Rₜ 1.98 min.

### Example 4

### Synthesis of Compound I (Third Route)

### 4-{(1S, 2S)-2-[((R)-4-Cyclobutyl-2-methylpiperazin-1-yl)carbonyl]-cyclo propyl}-benzamide

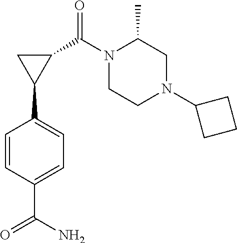

N₂ was bubbled into Intermediate P (6.09 g, 18.83 mmol) in EtOH (125 mL) and H₂O (30 mL) to this was added Hydrido(dimethylphosphinous acid-kP)[hydrogen bis(dimethylphosphnito-kP]platinum (II) (0.050 g, 0.12 mmol). The reaction was heated at reflux for 20 h. The reaction was heated for a further 24 h, concentrated to dryness and partitioned between ETOAc and H₂O. The aq. phase was extracted 3× with ETOAc, the combined organic layers were washed with brine, dried over Na₂SO₄, filtered and concentrated. The crude material was purified by flash chromatography on silica gel, eluting with a gradient of CH₂Cl₂ and MeOH, 2-10% with a plateau at 4% until elution of visible dark band followed by a second purification with a gradient of acetone/heptane 30-100% to afford 3.65 g Example 4 (56.8% yield) as a solid. ¹H NMR (400 MHz, Methanol-d₄) δ ppm 1.24 (br. s., 1H) 1.36 (br. s., 3H) 1.52-1.60 (m, 1H) 1.63-1.74 (m, 3H) 1.74-1.84 (m, 1H) 1.84-1.95 (m, 2H) 1.95-2.05 (m, 2H) 2.24 (br. s., 1H) 2.40 (br. s., 1H) 2.60-2.72 (m, 2H) 2.82 (d, J=12.50 Hz, 1H) 2.94 & 3.36 (t, J=12.11 Hz, 1H) 4.01 & 4.28 (d, J=13.28 Hz, 1H) 4.35 & 4.62 (br. s., 1H) 7.22 (d, J=8.20 Hz, 2H) 7.77 (d, J=8.59 Hz, 2H). The product was analyzed on analytical HPLC MS using the Zorbax gradient method (mobile phase: 5-95% B; A: H₂O with 0.05% TFA, B: CH₃CN, 4.5 min run) on Zorbax SB C18, 4.6×30 mm, 1.8 μm particle size. MS m/z 342.3 [M+H]⁺(ESI), Rₜ 0.584 min. The product was analyzed on chiral SFC (UV detection) using isocratic method (mobile phase: 55% EtOH with 0.1% DMEA, supercritical CO₂) on ChiralPak AD-H, 10×250 mm, 5 μm particle size, giving an enantiomeric purity of >99%, Rₜ 1.98 min. The title compound corresponds to “Isomer 1” of Example 1, above. HRMS m/z calcd for C₂₀H₂₇N₃O₂ 342.2176 [M+H]⁺, found 342.2176.

### EXAMPLE 5

### Preparation of Form I of Compound I

In a first means of preparing Form I of Compound I, 20 mg of an amorphous form of Compound I (prepared according to Example 1, 2 or 4 of the preceding synthetic routes) was added to a vessel. To the vessel, 100 μl of EtOAc was added to obtain a suspension. The resulting slurry was stirred at ambient temperature for 3 days. Solid crystalline material was then isolated and dried in air.

In a second means of preparing Form I of Compound I, 20 mg of an amorphous form of Compound I (prepared according to one of the preceding synthetic routes) was added to a vessel. To the vessel, 100 μl of ACN was added to obtain a suspension. The resulting slurry was stirred at ambient temperature for 3 days. Solid crystalline material was then isolated and dried in air.

### EXAMPLE 6

### Analysis of Form I of Compound I

Solid material obtained according to Example 5 was analyzed by XRPD. Selected peaks are provided in Table 1. A representative XRPD pattern is shown in FIG. 1. The XRPD pattern confirmed that the solid material was crystalline Form I of Compound I.

TABLE 1
Selected XRPD Peaks for Form I of Compound I

| Peak | 2θ | Intensity % |
|---|---|---|
| 1 | 5.3 | 60.9 |
| 2 | 8.5 | 47.3 |
| 3 | 10.6 | 20.3 |
| 4 | 15.5 | 18.2 |
| 5 | 16.3 | 42.3 |
| 6 | 18.0 | 100 |
| 7 | 18.4 | 34.2 |
| 8 | 19.3 | 68.2 |
| 9 | 20.9 | 36.3 |
| 10 | 21.4 | 37.3 |

Solid material obtained according to the Example 5 was analyzed by thermal techniques. DSC analysis indicated that Form I is a high melting solid with an endothermic onset of melting at about 133.5° C. and a peak at about 135.3° C., as shown in FIG. 2. TGA indicated that Form I of Compound I exhibited a mass loss of about 0.25% upon heating from about 20° C. to about 100° C., and exhibited a further mass loss of about 0.25% upon heating from about 100° C. to about 160° C. Thermal analysis indicated that Form I of Compound I does not contain substantial quantities of solvent or water. A representative DSC thermogram is shown in FIG. 2. A representative TGA thermogram is shown in FIG. 3.

Solid material obtained according to Example 5 was analyzed by DVS techniques. Isothermic DVS analysis was performed at about ambient temperature by increasing a sample of Form I of Compound I from about 0% RH to about 90% RH. The DVS analysis indicated that Form I of Compound I adsorbs less than 2% is (between about 1.2% and about 1.4%) water by mass between about 0% RH and about 90% RH. DVS analysis indicated that Form I is substantially nonhygroscopic. A representative DVS isotherm plot is shown in FIG. 4.

Solid material obtained according to Example 5 was analyzed by SS-NMR. The spectrum displayed peaks at the following ppm values: 171.0624; 144.1716; 131.7559; 127.5291; 60.4671; 54.5210; 52.9234; 51.5593; 50.7770; 45.9523; 45.0427; 40.7924; 28.5029; 24.5826; 23.7109; 18.1318; 15.7476; 15.2935; 14.3726; 13.6745; and 13.1087. A representative SS-NMR spectrum is shown in FIG. 5.

Solid material obtained according to Example 5 was analyzed by FT-IR and FT-Raman spectroscopy. A representative FT-IR spectrum (top) and FT-Raman spectrum (bottom) are shown in FIG. 6.

### Example 7

### Instruments and Techniques

### XRPD Analysis

XRPD analysis was performed using a Bruker D8 diffractometer, which is commercially available from Bruker AXS Inc.™ (Madison, Wis.). The XRPD spectra were obtained by mounting a sample (approximately 20 mg) of the material for analysis on a single silicon crystal wafer mount (e.g., a Bruker silicon zero background X-ray diffraction sample holder) and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5406 is angstroms (i.e., about 1.54 angstroms). The sample was exposed for 1 second per 0.02 degree 2-theta increment (continuous scan mode) over the range 2 degrees to 40 degrees 2-theta in theta-theta mode. The running time was 31 min, 41 s.

### DSC Analysis

DSC was performed using a TA Instruments model Q1000. A sample (approximately 2 mg) was weighed into an aluminium sample pan and transferred to the DSC. The instrument was purged with nitrogen at 50 mL/min and data collected between 25° C. and 300° C., using a dynamic heating rate of 10° C./min.

DSC analysis is performed on samples prepared according to standard methods using a Q SERIES™ Q1000 DSC calorimeter available from TA INSTRUMENTS® (New Castle, Del.). The instrument was purged with nitrogen at 50 mL/min and data collected between 25° C. and 300° C., using a dynamic heating rate of 10° C./minute. Thermal data is analyzed using standard software, e.g., Universal v.4.5A from TA INSTRUMENTS®.

### DVS Analysis

DVS analysis is performed on samples prepared according to standard methods using standard equipment, e.g., a DVS instrument commercially available from Surface Measurement Systems, Ltd.™(Alperton, London, UK). Samples maintained at ambient temperature are cycled between about 0% RH and about 90% RH. Percent changes in mass are recorded, which are indicative of moisture sorption and desorption.

### SS-NMR Analysis

Approximately 100 mg of material (e.g., drug substance or formulation) for analysis was packed into a 4 mm zirconium dioxide rotor sealed with a Kel-F cap. For determination of ¹³C Cross Polarization Magic Angle Spinning spectra the rotor was spun typically between 5 and 9 kHz (to remove chemical shift anisotropy) and the ¹³C spectrum was recorded using cross polarization from hydrogen (to improve sensitivity and reduce experiment times). The contact time for the magnetization transfer was typically 2 milliseconds and the inter-pulse delay (allowing for nuclear relaxation) was typically 5 sec. Signal averaging was employed with sufficient scans recorded to enable all the major peaks to be resolved from the noise. Typical experiment times for a crystalline drug substance were about 1 h.

### FT-IR and FT-Raman Analysis

FT-IR/ATR spectrum is collected using Thermo Nicolet Nexus 870 equipped with DTGS KBr detector over the range of 400 to 4000 cm−1 with a resolution of 4 cm−1 and scan numbers of 64. The crystal used in the ATR is a diamond.

TABLE 2
FT-IR For Form I of Compound I

| Transmission (cm−1) | Intensity |
|---|---|
| 3378.97 | 0.731 |
| 3171.70 | 0.725 |
| 2939.02 | 0.638 |
| 2808.65 | 0.505 |
| 1646.80 | 0.774 |
| 1607.63 | 1.32 |
| 1567.34 | 0.545 |
| 1414.45 | 0.701 |
| 1234.13 | 0.576 |
| 1055.18 | 0.432 |
| 798.42 | 0.319 |

FT-Raman spectrum is collected on Thermo Nicolet Nexus 870 equipped with InGaAs dtector over the range of 100 to 3700 cm−1 with a resolution of 8 cm−1 and scan numbers of 64. Data acquisition and analysis were performed using Thermo Nicolet software Omnic software.

TABLE 3
FT-Raman for Form I of Compound I

| Raman Shift (cm−1) | Intensity |
|---|---|
| 3070.22 | 4.905 |
| 3006.28 | 5.919 |
| 2940.36 | 6.904 |
| 2867.12 | 2.688 |
| 2808.64 | 2.533 |
| 2767.97 | 2.263 |
| 1614.44 | 26.926 |
| 1562.48 | 4.593 |
| 1219.17 | 6.195 |
| 1144.15 | 7.002 |

### Intermediate A

### 4-(trans-2-((R)-2-methylpiperazine-1-carbonyl)cyclopropyl)benzamide

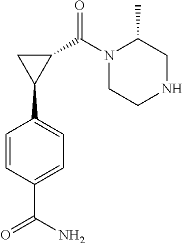

Intermediate B (849 mg, 2.19 mmol) was dissolved in DCM (10.0 mL). TFA (5.00 mL) was added and the reaction mixture stirred at rt for 30 min. Volatiles were evaporated under reduced pressure to give a yellow gum. The crude material was used in the next step without purification. ¹H NMR (400 MHz, CD₃OD) δ ppm 1.33 (d, J=7.03 Hz, 3H) 1.37-1.52 (m, 3H) 1.65 (br. s., 1H) 2.26-2.39 (m, 1H) 2.51 (br. s., 1H) 3.11 (br. s., 1H) 3.21-3.45 (m, 4H) 7.27 (d, J=8.20 Hz, 2H) 7.81 (d, J=8.20 Hz, 2H).

### Intermediate B

### (R)-tert-butyl-4-(trans-2-(4-carbamoylphenyl)cyclopropanecarbonyI)-3-methylpiperazine-1-carboxylate

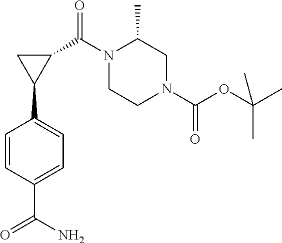

Intermediate C (450 mg, 2.19 mmol) was dissolved in DMF (20 mL). DIPEA (1.149 mL, 6.58 mmol) was added, followed by HOBT (444 mg, 3.29 mmol), EDC (631 mg, 3.29 mmol) and Intermediate D (527 mg, 2.63 mmol). The reaction mixture was stirred at rt for 2 days, concentrated under reduced pressure, redissolved in EtOAc, washed with 1M HCl and sat. NaHCO₃, dried over MgSO₄, filtered and concentrated under reduced pressure to give Intermediate B as a solid. The crude product was used in the next step without further purification. MS m/z 388.34 [M+H]⁺ (ESI).

### Intermediate C

### trans-2-(4-carbamoylphenyl)cyclopropanecarboxylic acid

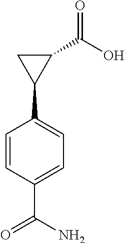

Intermediate E (3.4 g, 18.16 mmol) was dissolved in t-BuOH (90 mL). Grounded KOH (5.10 g, 90.81 mmol) was added, the reaction mixture was heated to 70° C. overnight, cooled to rt and concentrated under reduced pressure. The residue was redissolved in H₂O and washed with EtOAc. The aq. phase was acidified to pH 4-5 with 1 M HCl. The precipitate was filtered and dried under vacuum to give 3.06 g Intermediate C (82%) as a solid. The product was used in the next step without further purification. ¹H NMR (400 MHz, CD₃OD) δ ppm 1.42 (ddd, J=8.50, 6.35, 4.69 Hz, 1H) 1.55-1.62 (m, 1H) 1.91 (ddd, J=8.50, 5.37, 4.10 Hz, 1H) 2.52 (ddd, J=9.18, 6.25, 4.10 Hz, 1H) 7.20-7.26 (m, 2H) 7.76-7.83 (m, 2H); MS m/z 206.22 [M+H]⁺ (ES+).

### Intermediate D

### (R)-tert-butyl 3-methylpiperazine-1-carboxylate

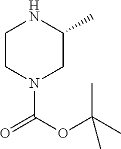

(R)-2-methylpiperazine (5.025 g, 50.2 mmol) was dissolved in DCM (100 mL). A solution of boc anhydride (5.47 g, 25.1 mmol) in DCM (50 mL) was added dropwise at 0° C. The reaction mixture was stirred at rt for 1 h. The solution was filtered and concentrated under reduced pressure. H₂O (100 mL) was added to the residue, which was filtered again. The filtrate was saturated with K₂CO₃ and extracted with Et₂O (3×150 mL). The combined organic layers were dried over anhydrous Na₂SO₄, filtered and concentrated under reduced pressure to provide 5.04 g Intermediate D (50%) as a solid. ¹H NMR (300 MHz, CDCl₃) δ ppm 1.03 (d, J=6.3 Hz, 3H) 1.45 (s, 9H) 1.56 (s, 1H) 2.30-2.46 (m, 1H) 2.65-2.72 (m, 1H) 2.74-2.76 (m, 2H) 2.93-2.95 (m, 1H) 3.93 (br s, 2H). Intermediate D is also commercially available from Lanzhou Boc Chemical Co.

### Intermediate E (First Method)

### trans-2-(4-cyanophenyl)cyclopropanecarboxylic acid

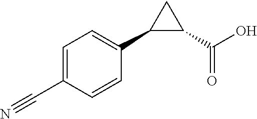

Intermediate H (11.2 g, 64.7 mmol) was dissolved in acetone (100 mL). The solution was cooled to −10° C. Jones reagent (65 mL) was added over a period of 30 min. After completing addition, the reaction was warmed to rt and then quenched by adding 2-propanol (100 mL). The resulting mixture was diluted with EtOAc (200 mL). MgSO₄ was added and stirring was continued for another 30 min. The mixture was filtered and the filtrate was concentrated under reduced pressure. The residue was redissolved in EtOAc (200 mL), washed with 2×75 mL of H₂O, dried over MgSO₄, filtered and concentrated under reduced pressure. The crude was purified by trituration with EtOAc (20 mL) to afford 5.2 g Intermediate E (43%) as a solid. ¹H NMR (400 MHz, DMSO-d₆) δ ppm 1.39-1.46 (m, 1H) 1.47-1.55 (m, 1H) 1.90-1.98 (m, 1H) 2.45-2.55 (m, 1H) 7.38 (d, J=8.2 Hz, 2H) 7.73 (d, J=8.2 Hz, 2H).

Preparation of Jones reagent: Jones reagent was prepared by dissolving 26.7 g of CrO₃ in 23 mL concentrated H₂SO₄ and diluting the mixture to 100 mL with H₂O.

### Intermediate E (Second Method)

### trans-2-(4-cyanophenyl)cyclopropanecarboxylic acid

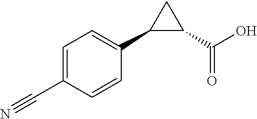

Intermediate F (11.6 g, 47.7 mmol) was dissolved in MeOH (55 mL). A solution of NaOH (5.7 g, 143.1 mmol) in H₂O (30 mL) was added and the resultant mixture was heated at 70° C. for 4 h. After cooling to rt, the mixture was concentrated to one-third its volume and diluted by the addition of 50 mL of 0.5 M NaOH. The resultant mixture was washed with 2×25 mL of MTBE. The aq. layer was separated and acidified by addition of concentrated HCl until pH 1. The acidified aq. phase was extracted with 2×50 mL of EtOAc. The combined organic extracts were dried over MgSO₄, filtered and evaporated to dryness. The crude was purified by flash chromatography (silica, DCM:MeOH 99:1 to 90:10), giving 3.1 g Intermediate E (36.4%) as a solid. ¹H NMR (400 MHz, CDCl₃) δ ppm 1.37-1.46 (m, 1H) 1.47-1.55 (m, 1H) 1.87-1.98 (m, 1H) 2.43-2.49 (m, 1H) 7.38 (d, J=8 Hz, 2H) 7.74 (d, J=8 Hz, 2H) 12.43 (s, 1H).

### Intermediate F

### trans-tert-butyl 2-(4-cyanophenyl)cyclopropanecarboxylate

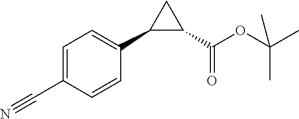

Trimethylsulfoxonium iodide (37.9 g, 172.4 mmol) was dissolved in DMSO (450 mL) under nitrogen. Sodium tert-butoxide (16.5 g, 172.4 mmol) was added and the resultant mixture was stirred at rt for 2 h. Intermediate G (20 g, 86.2 mmol) as added and the reaction mixture was stirred at rt for 16 h. The reaction mixture was diluted by sequential addition of MTBE (500 mL) and brine (300 mL). The organic layer was separated, dried over MgSO₄, filtered and evaporated to dryness. The crude product was purified by flash chromatography (silica, heptane/EtOAc 95:5 to 90:10), giving 11.6 g Intermediate F (54%) as a solid. ¹H NMR (400 MHz, CDCl₃) δ ppm 1.29-1.23 (m, 1H) 1.49 (s, 9H) 1.57-1.69 (m, 1H) 1.83-1.96 (m, 1H) 2.40-2.53 (m, 1H) 7.18 (d, J=8 Hz, 2H) 7.57 (d, J=8 Hz, 2H).

### Intermediate G

### (E)-tert-butyl 3-(4-cyanophenyl)acrylate

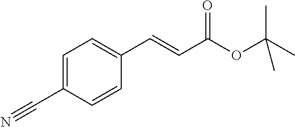

A flame-dried three-neck round-bottom flask equipped with a magnetic stirring bar, a thermometer, an addition funnel and a nitrogen inlet was charged with NaH (3.96 g, 94.7 mmol) and anhydrous THF (120 mL). Tertbutyldiethylphosphono acetate (23.2 mL, 94.7 mmol) dissolved in anhydrous THF (20 mL) was added dropwise via the addition funnel over a period of 30 min. After the completion of addition, the reaction mixture was stirred at rt for another 30 min. A solution of 4-cyanobenzaldehyde (11.3 g, 86.1 mmol) dissolved in anhydrous THF (20 mL) was added to the reaction mixture dropwise via the addition funnel over a period of 30 min. After the end of the addition, the reaction mixture was stirred at rt for 1 h, then diluted with MTBE (200 mL) and sat. NH₄Cl (150 mL). The organic layer was separated, washed with 25 mL of H₂O and 25 mL of sat. NH₄Cl, dried over MgSO₄, filtered and evaporated to dryness to give 20.0 g Intermediate Gas a solid (100%). ¹H NMR (400 MHz, CDCl₃) δ ppm 1.56 (s, 9H) 6.47 (d, J=16 Hz, 1H) 7.58 (d, J=16 Hz, 1H) 7.61 (d, J=8 Hz, 2H) 7.68 (d, J=8 Hz, 2H).

### Intermediate H

### trans-4-(2-(hydroxymethyl)cyclopropyl)benzonitrile

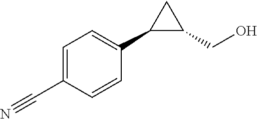

A round bottom flask was charged with Intermediate I (10.0 g, 44 mmol), dimethylacetamide (125 mL), potassium hexacyanferrate (II) trihydrate (24.2 g, 22 mmol), palladium (II) acetate (1.3 g, 2.2 mmol), DABCO (1.3 g, 4.4 mmol), and sodium carbonate (12.2 g, 44 mmol). The resulting mixture was heated to 150° C. under nitrogen for 17 h. The reaction mixture was cooled to rt and filtered through a pad of silica gel. The pad was washed with EtOAc (200 mL). The combined filtrate and washing were diluted with more EtOAc (200 mL), washed with brine (3×100 mL), dried over MgSO₄, filtered and concentrated under reduced pressure. The crude was purified by column chromatography (silica, DCM/MeOH 99:1) to give 10.5 g Intermediate H (55%). ¹H NMR (400 MHz, CDCl₃) δ ppm 1.00-1.15 (m, 2H) 1.47-1.58 (m, 1H) 1.88-1.94 (m, 1H) 3.56-3.76 (m, 2H) 7.15 (d, J=8.5 Hz, 2H) 7.55 (d, J=8.5 Hz, 2H).

### Intermediate I

### trans-2-(4-bromophenyl)cyclopropyl)methanol

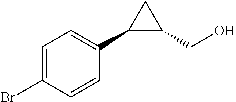

A solution of diethyl zinc (1.1 M, 695 mL, 765 mmol) in hexanes was added to a flame-dried 3-necked round bottom flask containing 450 mL of DCM under nitrogen. The resulting solution was cooled to 0-5° C. TFA (59 mL, 765 mmol) was added slowly to the cooled diethylzinc solution. After the completion of addition, the resulting mixture was stirred for 20 min. A solution of CH₂I₂ (62 mL, 765 mmol) in 50 mL of DCM was added to the mixture. After an additional 20 min of stirring, a solution of 3-(4-bromophenyl)prop-2-en-1-ol (81.6 g, 382.9 mmol) in 450 mL of DCM was added. After completing addition, the reaction mixture was warmed to rt and stirred for 2 h. Excess reagent was quenched by slow addition of 500 mL of 1 M HCl. The top aq. layer was separated and extracted with 200 mL of DCM. The combined organic extracts were washed with 500 mL of a mixture of sat. NH₄Cl and NH₄OH (9:1 v/v), dried over MgSO₄, filtered and concentrated under reduced pressure. The crude was purified by flash column chromatography (silica, heptane/EtOAc 10:1), giving 76.1 g Intermediate I as a solid (87.5%). ¹H NMR (400 MHz, CDCl₃) δ ppm 0.90-1.00 (m, 2H) 1.36-1.48 (m, 1H) 1.75-1.85 (m, 1H) 3.62 (t, J=6 Hz, 2H) 6.95 (d, J=8.5 Hz, 2H) 7.38 (d, J=8.5 Hz, 2H).

### Intermediate J

### (R)-1-(4-Bromo-phenyl)-2-chloro-ethanol

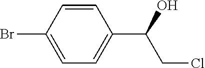

Borane dimethylsulfide (2.0 kg, 24.8 moles, 94% w/w) was mixed in toluene (8 L) at t_{jacket}=20° C. (R)-(+)-Methyl-CBS-oxazaborolidine (2.6 kg, 2.74 moles, 1 M) as a toluene solution was added. The charging vessel was rinsed with toluene (0.5 L) and t_{jacket} was set to 45° C. 1-(4-Bromo-phenyl)-2-chloro-ethanone (7.84 kg, 33.6 moles), which is commercially available from Jiangyan Keyan Fine Chemical Co. Ltd, was dissolved in 2-MeTHF (75 L) in a separate vessel and when tᵢₙₙₑᵣ was above 40° C. in the first vessel, the 2-MeTHF solution was added during 3 h. The latter vessel was rinsed with 2-MeTHF (2 L) and added to the reaction mixture, which was left stirring at t_{jacket}=45° C. for 1 h. Analysis of a sample on HPLC indicated full conversion at this point using the following gradient method (mobile phase 20-95% B; A: 5% CH₃CN in H₂O with 0.1% TFA, B: 95% CH₃CN in H₂O with 0.085% TFA, 10 min run) on Chromolith Performance RP-18e, 4.6×100 mm. The reaction mixture was cooled to t_{jacket}=10° C. before slow quench with MeOH (36 L). The first liter of MeOH was added during 30 min. and the rest during additional 30 min. MeOH was distilled off under vacuum at t_{jacket}=50° C. The organic solution left was cooled to t_{jacket}=20° C., washed with 1M HCl in H₂O (7 L conc HCl+73 L H₂O) and concentrated under vacuum at t_{jacket}=50° C. to approximately 40 L. Intermediate J obtained in a 2-MeTHF solution can be stored at 10° C. for 20 h or used directly in the next synthetic step.

### Intermediate K

### (R)-2-(4-Bromo-phenyl)-oxirane

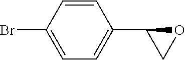

Aliquat® 175 (methyl tributyl ammonium chloride) (1.12 kg, 4.75 moles) was added to Intermediate J as a 2-MeTHF solution (33.6 moles, 40 L) at t_{jacket}=20° C. NaOH (5.1 kg, 57.4 moles, 45% w/w) diluted in H₂O (2 L) was added during 20 min. The reaction mixture was left stirring at t_{jacket}=20° C. for 2 h. Analysis of a sample on HPLC indicated full conversion at this point using the following gradient method (mobile phase 20-95% B; A: 5% CH₃CN in H₂O with 0.1% TFA, B: 95% CH₃CN in H₂O with 0.085% TFA, 10 min run) on Chromolith Performance RP-18e, 4.6×100 mm. The aq. phase was separated off and the organic phase washed with H₂O (2×25 L). 2-MeTHF (25 L) was added and the organic phase concentrated under vacuum at t_{jacket}=50° C. to approximately 30 L. Intermediate K obtained in a 2-MeTHF solution, can be stored at 5° C. for 140 h or used directly in the next synthetic step.

### Intermediate L

### (1S, 2S)-2-(4-Bromo-phenyl)-cyclopropanecarboxylic acid

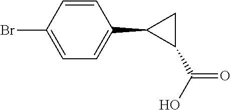

Triethyl phosphonoacetate (10.5 L, 51.9 moles, 98% w/w) was dissolved in 2-MeTHF (14 L) at t_{jacket}=−20° C. Hexyl lithium in hexane (21 L, 48.3 moles, 2.3 M) was added at a rate to maintain tᵢₙₙₑᵣ below 0° C. The charging vessel was rinsed with 2-MeTHF (3 L) and the reaction solution was left stirring at t_{jacket}=10° C. Intermediate K as a 2-MeTHF solution (33.6 moles, 30 L) was added during 20 min. The charging vessel was rinsed with 2-MeTHF (2 L) and the reaction solution was left stirring at t_{jacket}=65° C. for at least 16 h with the last 3 h at t_{jacket}=75° C. Analysis of a sample on HPLC using the following gradient method (mobile phase 20-95% B; A: 5% CH₃CN in H₂O with 0.1% TFA, B: 95% CH₃CN in H₂O with 0.085% TFA, 10 min run) on Chromolith Performance RP-18e, 4.6×100 mm indicated full conversion to the intermediate (1S, 2S)-2-(4-bromo-phenyl)-cyclopropanecarboxylic acid ethyl ester. The reaction solution was cooled to t_{jacket}=20° C. NaOH (7.6 kg, 85.5 moles, 45% w/w) diluted in H₂O (12 L) was added over 20 min. The reaction solution obtained was left stirring at t_{jacket}=60° C. for at least 2 h. Analysis of a sample on HPLC indicated full conversion at this point using the following gradient method (mobile phase 20-95% B; A: 5% CH₃CN in H₂O with 0.1% TFA, B: 95% CH₃CN in H₂O with 0.085% TFA, 10 min run) on Chromolith Performance RP-18e, 4.6×100 mm. The reaction solution was cooled to t_{jacket}=20° C., the aq. phase was separated off and the organic phase was extracted with H₂O (37 L). The combined aq. phases were acidified to pH<3.5 with H₃PO₄ (9 L, 131 moles, 85% w/w) diluted in H₂O (12.5 L). Only 17 L of the diluted H₃PO_{4 (aq)} was used to achieve the pH<3.5. The acidic aq. phase was extracted with 2-MeTHF (2×15 L). The combined organic phases including rinsing with 2-MeTHF (2 L) were concentrated under vacuum at t_{jacket}=50° C. to approximately 11 L. The 2-MeTHF solution was diluted with EtOH (14.5 L) at t_{jacket}=35° C. and H₂O (16 L) was added over 20 min. The reaction solution was cooled to t_{jacket}=28° C. Seed (16 g, 0.066 moles) was added and the solution was stirred for 2 h at t_{jacket}=28° C. The reaction mixture was cooled to t_{jacket}=0° C. over 6 h and left stirring for at least 1 h. Additional H₂O (8 L) was added during 40 min. and the product was filtered off and washed with cold H₂O (10 L). Drying under vacuum at 40° C. gave 6.18 kg Intermediate L (21.5 moles, 84% w/w), 64% yield over four steps from 7.84 kg 1-(4-bromo-phenyl)-2-chloro-ethanone (33.6 moles).

Recrystallization of Intermediate L: Two batches of Intermediate L (6.18+7.04 kg) were mixed in EtOH (52 L) and heated at t_{jacket}=70° C. H₂O (52 L) was added. The reaction solution was cooled to t_{jacket}=30° C. over 2.5 h. H₂O (16 L) was added during 20 min. and the crystallization was cooled to t_{jacket}=20° C. during 3 h. The product was filtered off and washed with a mixture of H₂O (8 L) and EtOH (2 L). Drying under vacuum at 40° C. gave 10.0 kg Intermediate L (41.5 moles, 88% w/w), which was redissolved in toluene (39 L) and isooctane (57 L) at t_{jacket}=60° C. A clear solution was obtained. The reaction solution was cooled to t_{jacket}=45° C. and left stirring for 1 h, then cooled to t_{jacket}=20° C. over 2 h. The product was filtered off and washed with a mixture of toluene (4 L) and isooctane (36 L) in two portions. Drying under vacuum at 40° C. gave 7.4 kg Intermediate L (29.8 moles, 97% w/w), 44% yield over four steps from 7.84+7.93 kg 1-(4-bromo-phenyl)-2-chloro-ethanone (67.5 moles). ¹H-NMR (DMSO-d₆): δ 12.36 (s, 1H), 7.44 (d, 2H, J=8 Hz), 7.13 (d, 2H, J=8 Hz), 2.39 (m, 1H), 1.81 (m, 1H), 1.43 (m, 1H), 1.33 (m, 1H); ¹³C-NMR (DMSO-d₆): δ 173.76, 139.88, 131.20, 128.24, 119.14, 24.73, 24.31, 16.78; LC-MS (ES): m/z 239 (M-1 (Br⁷⁹)) and 241 (M-1 (Br⁸¹)). Rₜ=5.03 min with the analytical method (mobile phase: 5-90% B; A: H₂O with 0.1% formic acid, B: CH₃CN, 8.6 min run) on Xbridge C18, 3.0×50 mm, 2.5 μm particle size. The product was analyzed on a chiral column with UV-detection using isocratic method (mobile phase: EtOH/Isohexane/TFA (15/85/0.1 v/v/v)) on Kromosil 3-Amycoat, 150×4.6 mm, 3 μm particle size, giving an enantiomeric purity of 98.9% ee, Rₜ=5.29 min (isomer 1) and 5.97 min (isomer 2).

### Intermediate M

### (1S, 2S)-2-(4-Cyano-phenyl)-cyclopropanecarboxylic acid

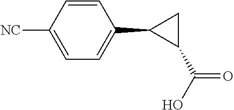

Intermediate L (3.7 kg, 14.9 moles, 97% w/w) and zinc-dust (98%+, <10 μm) (99 g, 1.51 moles) were mixed with DMF (13.5 L) and the slurry was stirred at t_{jacket}=20° C. The mixture was inerted and left with N₂ pressure of 0.1-0.2 bar. Bis(tri-t-butylphosphine)palladium (0) (27.5 g, 0.054 moles) was added to the slurry, and the vessel was inerted and left with N₂ pressure of 0.1-0.2 bar. The mixture was heated to t_{jacket}=45° C., Zn(CN)₂ (1.0 kg, 8.52 moles) was added to the suspension in one portion, and the system was inerted and left with N₂ pressure of 0.1-0.2 bar (N.B. Cyanide salts are highly toxic). The resulting mixture was heated to t_{jacket}=75° C. and stirred for at least 2 h. Analysis of a sample on HPLC indicated full conversion at this point using the following gradient method (mobile phase 20-95% B; A: 5% CH₃CN in H₂O with 0.05% formic acid, B: 95% CH₃CN in H₂O with 0.05% formic acid, 8 min run) on Chromolith Performance RP-18e, 4.6×100 mm. The reaction mixture was cooled to t_{jacket}=20° C. Thiol-functionalized silica (Silicycle, SiliaBond Thiol) (1.07 kg, 28% w/w) was added and the vessel was inerted. The reaction mixture was stirred for at least 36 h at t_{jacket}=20° C. The scavenger was filtered off via a filter with activated charcoal or equivalent (pall-filter). The vessel and the filter system were washed with 2-MeTHF (53 L). The filtrate and washings were combined and stirred at t_{jacket}=5° C. A pale yellow liquid resulted. NaCl (3.5 kg) in H₂O (16.4 L) was added during 15 min. at such a rate so the inner temperature remained below 15° C. The resulting reaction mixture was heated to t_{jacket}=45° C. and the aq. phase was separated off. The organic phase was washed with NaHSO₄×H₂O in H₂O (2×(2.87 kg+16.4 L)) and NaCl in H₂O (3.5 kg +16.4 L). The organic phase was cooled to t_{jacket}=10° C. and NaOH (1.54 kg, 19.3 moles, 50% w/w) diluted in H₂O (41 L) was added during 45 min. The resulting reaction mixture was heated to t_{jacket}=30° C. and the organic phase was separated off. The aq. phase was stirred at t_{jacket}=20° C. and pH adjusted to 6.5 with H₃PO₄ (0.90 kg, 7.81 moles, 85% w/w) diluted in H₂O (5.3 L) at a rate that maintained the inner temperature below 25° C. 2-MeTHF and H₂O were distilled off under vacuum until a volume 85-90% of the volume prior to distillation, approximately 8 L. The reaction mixture was cooled to t_{jacket}=0° C. and continued charging off H₃PO₄ (1.17 kg, 10.1 moles, 85% w/w) diluted in H₂O (8.2 L) until pH=4. The slurry was left stirring overnight at t_{jacket}=10° C. The product was filtered off, washed with H₂O (2×4 L). Drying under vacuum at 40° C. gave Intermediate M (2.24 kg, 11.2 moles, 93.2% w/w), 75% yield. ¹H-NMR (DMSO-d₆): δ 12.45 (s, 1H), 7.72 (d, 2H, J=8 Hz), 7.37 (d, 2H, J=8 Hz), 2.50 (m, 1H), 1.94 (m, 1H), 1.50 (m, 1H), 1.42 (m, 1H); ¹³C-NMR (DMSO-d₆): δ 173.51, 146.68, 132.27, 126.93, 118.97, 108.85, 25.16, 25.04, 17.44; LC-MS (ESI): m/z 186 (M-1). Rₜ=3.63 min with the analytical method (mobile phase: 5-90% B; A: H₂O with 0.1% formic acid, B: CH₃CN, 8.6 min run) on Xbridge C18, 3.0×50 mm, 2.5 μm particle size.

### Intermediate N

### (1S, 2S)-2-(4-Carbamoyl-phenyl)-cyclopropanecarboxylic acid

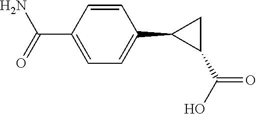

Intermediate M (4.46 kg, 22.0 moles, 92.5% w/w) was mixed in H₂O (40 L) at t_{jacket}=30° C. NaOH (2.25 kg, 28.1 moles, 50% w/w) diluted in H₂O (6 L) was added at such a rate so tᵢₙₙₑᵣ remained below 35° C. The charging vessel was rinsed with H₂O (1 L). If the pH was not ≧12, more NaOH was charged in the same concentration as previously. Hydrogen peroxide (4.89 kg, 50.3 moles, 35% w/w) was added at a rate to maintain tᵢₙₙₑᵣ below 35° C. The charging vessel was rinsed with H₂O (1 L) and the reaction slurry was left stirring for 0.5-1.0 h. Analysis of a sample on HPLC indicated full conversion at this point using the following gradient method (mobile phase 20-95% B; A: 5% CH₃CN in H₂O with 0.05% formic acid, B: 95% CH₃CN in H₂O with 0.05% formic acid, 8 min run) on Chromolith Performance RP-18e, 4.6×100 mm. The reaction mixture was cooled to t_{jacket}=0° C. and left stirring for at least 0.5 h when the temperature was reached. The sodium salt of Intermediate N was filtered off and washed with cold H₂O (2×7 L). The solid was slurry washed on the filter with NaHSO₄×H₂O (2.76 kg, 20.0 moles) diluted in H₂O (35 L). The slurry was kept stirring at t_{jacket}=0° C. for 1 h. If the pH was not <3.7, it was adjusted with NaHSO₄×H₂O in H₂O. The product was filtered off, washed with cold H₂O (3 ×14 L). Drying under vacuum at 40° C. gave 4.0 kg Intermediate N (18.2 moles, 93.4% w/w), 83% yield. ¹H-NMR (DMSO-d₆): δ 12.40 (s, 1H), 7.94 (s, 1H), 7.79 (d, 2H, J=8 Hz), 7.32 (s, 1H), 7.23 (d, 2H, J=8 Hz), 2.44 (m, 1H), 1.88 (m, 1H), 1.47 (m, 1H), 1.39 (m, 1H); ¹³C-NMR (DMSO-d₆): δ 173.83, 167.67, 143.94, 132.17, 127.68, 125.73, 25.21, 24.67, 17.11; LC-MS (ESI): m/z 206 (M+1). Rₜ=2.13 min with the analytical method (mobile phase: 5-90% B; A: H₂O with 0.1% formic acid, B: CH₃CN, 8.6 min run) on Xbridge C18, 3.0×50 mm, 2.5 μm particle size. The product was analyzed on a chiral column with UV-detection using isocratic method (mobile phase: EtOH/Isohexane/TFA (15/85/0.1 v/v/v)) on Kromosil 3-Amycoat, 150×4.6 mm, 3 μm particle size, giving an enantiomeric purity of >99% ee, Rₜ=13.40 min (isomer 1) and 22.22 min (isomer 2).

### Intermediate O

### (R)-1-Cyclobutyl-3-methylpiperazine×2HCl

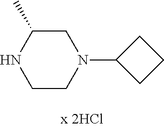

(R)-Boc-2-methylpiperazine (350 g, 1.71 moles, 98% w/w), which is commercially available from Lanzhou Boc Chemical Co., was dissolved in EtOH (2.75 L) at t_{jacket}=20° C. Acetic acid (1.37 L) was added in one portion followed by the addition of cyclobutanone (184 g, 2.57 moles). The charging vessel was rinsed with EtOH (250 mL) and the light yellow solution was left stirring at t_{jacket}=20° C. for 1 h. NaBH(OAc)₃ (497 g, 2.48 moles, 95% w/w) was added in 20 portions over 90 min. EtOH (340 mL) was used for rinsing. The reaction mixture was left stirring for 2 h. A sample was analyzed on GC using HP-5MS column (length 25 m, ID 0.32 mm, Film 0.52 μm) with a gradient method (2 min at 60° C., followed by 25° C./min during 8 min then 2 min at 260° C.). Frontinlet temperature=200° C. using He as gas and a detector temperature=300° C. More NaBH(OAc)₃ (30 g, 0.14 moles) was added to complete the reaction within 1 h. The reaction mixture was cooled to t_{jacket}=0° C. before quenching with 5M NaOH (5.5 L). EtOH was distilled off under vacuum at t_{jacket}=50° C. The H₂O phase was extracted with toluene (5.5 L) at t_{jacket}=20° C. The organic phase was combined with a second batch, started with (R)-Boc-2-methylpiperazine (300 g, 1.47 moles, 98% w/w). The combined organic phases were concentrated under vacuum at t_{jacket}=50° C. to approximately 2 L. The obtained toluene solution with the intermediate can be stored at 5° C. for several days. The is toluene solution was diluted with 2-propanol (2 L) at t_{jacket}=10° C., and HCl in 2-propanol (1.06 L, 6.36 moles, 6M) diluted in 2-propanol (2 L) was added over 30 min. The reaction solution was heated to t_{jacket}=48° C. HCl in 2-propanol (2.12 L, 12.72 moles, 6M) diluted in 2-propanol (2 L) was added over 2 h at tᵢₙₙₑᵣ=46° C. The reaction solution was kept at t_{jacket}=48° C. for an additional 3 h before being cooled to t_{jacket}=0° C. over 1 h. A seed mixture (0.4 L reaction solution with Intermediate O (0.2 g, 0.89 mmoles)) was added. The reaction mixture was left stirring at t_{jacket}=0° C. overnight and the product was filtered off. Drying under vacuum at 40° C. gave 620 g Intermediate O (2.63 moles, 96.3% w/w), 83% yield. ¹H-NMR (DMSO-d₆): δ 12.46 (s, 1H), 10.13 (s, 2H), 3.35-3.74 (m, 6H), 3.09 (m, 1H), 2.92 (m, 1H), 2.39 (m, 2H), 2.16 (m, 2H), 1.72 (m, 2H), 1.32 (d, 3H, J=6.4 Hz); ¹³C-NMR (DMSO-d₆): δ 58.50, 49.62, 48.13, 44.30, 24.48, 24.38, 15.25, 13.26.

### Intermediate P

### 4-((1S, 2S)-2-((R)-4-cyclobutyl-2-methylpiperazine-1-carbonyl)cyclopropyl)benzonitrile

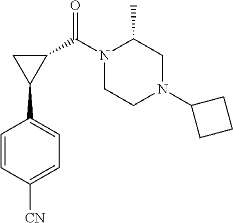

To a solution of Intermediate Q (8.5 g, 22.53 mmol) in NMP (100 mL) while bubbling Ar was added Zinc (0.737 g, 11.26 mmol), Zinc cyanide (1.984 g, 16.90 mmol) and dichloro[1,1′-bis(di-t-butylphosphino)ferrocene]palladium(II) (0.335 g, 0.45 mmol). This was heated at 100° C. for 20 h. Some starting material was still present, therefore heating was continued for further a 24 h and the reaction then cooled and concentrated under high vac. The material was taken into EtOAc and filtered through celite. The filtrate was concentrated, divided into two portions of equal weight, wherein each portion was purified on a 120 g silica gel column eluting with a gradient of EtOAc/heptane 50-100% providing 6.10 g Intermediate P (84%). The product was analyzed on analytical HPLC MS using the high pH gradient method (mobile phase: 5-95% B; A: H₂O with 10 mM NH₄CO₃ and 0.375% NH₄OH v/v, B: CH₃CN, 2.25 min run) on X-Bridge C18, 2.1×30 mm, 5 μm particle size. MS m/z 324.39 [M+H]⁺ (ESI), Rₜ 1.76 min.

### Intermediate Q

### ((1S,2S)-2-(4-bromophenyl)cyclopropyl)((R)-4-cyclobutyl-2-methylpiperazin-1-yl)methanone

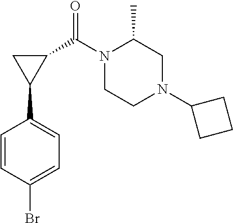

To a solution of Intermediate R (second method) (5.87 g, 24.34 mmol) in DMF (120 mL) at 0° C. was added N,N-Diisopropylethylamine (21.20 mL, 121.72 mmol), 1-Hydroxybenzotriazole (4.93 g, 36.52 mmol), N-(3-Dimethylaminopropyl)-N′-ethylcarbodiimide hydrochloride (7 g, 36.52 mmol) followed by Intermediate O (5.53 g, 24.34 mmol). The reaction was stirred for 15 h then the reaction was concentrated and the residue taken into EtOAc and washed with a sat. solution of NaHCO₃. The aq. phase was extracted twice with EtOAc and the combined organics were washed with brine, dried over MgSO₄, filtered and concentrated. The resulting oil was purified by normal phase chromatography using a gradient of EtOAc/Heptane 20-100% on a 120 g Redisep column using an ISCO Companion instrument providing 8.50 g Intermediate Q (93%) as clear glass that solidified slowly on standing. ¹H-NMR (400 MHz, Methanol-d₄) δ ppm 1.27 (br. s., 3H) 1.38 (br. s., 1H) 1.48-1.58 (m, 1H) 1.64-1.77 (m, 3H) 1.77-1.87 (m, 1H) 1.87-1.99 (m, 2H) 1.98-2.09 (m, 2H) 2.14-2.22 (m, 1H) 2.34 (br. s., 1H) 2.63-2.76 (m, 2H) 2.85 (dddd, J=11.43, 3.61, 1.95, 1.76 Hz, 1H) 2.90-3.01 (m, 1H) 3.40 (br. s., 1H) 4.03 (d, J=11.33 Hz, 1H) 4.31 (d, J=11.72 Hz, 1H) 4.39 (br. s., 1H) 4.64 (br. s., 1H) 7.09 (d, J=8.20 Hz, 7.41 (d, J=8.59 Hz, 2H). The product was analyzed on analytical HPLC MS using the high pH gradient method (mobile phase: 5-95% B; A: H₂O with 10 mM NH₄CO₃ and 0.375% NH₄OH v/v, B: CH₃CN, 2.25 min run) on X-Bridge C18, 2.1×30 mm, 5 μm particle size. MS m/z 277.31 [M+H]⁺ (ESI), Rₜ 2.10 min.

### Intermediate R

### (1S, 2S)-2-(4-Bromo-phenyl)-cyclopropanecarboxylic acid

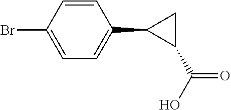

To a stirred solution of (trans)-2-(4-bromophenyl)cyclopropanecarboxylic acid (6.52 g, 27.04 mmol), which can be prepared in accordance with the process set forth on page 82 of WO 2009/024823, in 400 ml of EtOH was added a solution of (R)-(+)-1-(1-Naphthyl)ethylamine (4.63g, 4.37 mL, 27.04 mmol), in 100 ml of EtOH followed by 25 ml of H₂O. This was stirred at rt for about 4 h. The solid was collected by filtration and washed with 40 ml of cold EtOH/H₂O (20/1) providing 3.18 grams of salt as a white solid (58% recovery) equivalent to 1.86 g of free acid. This was taken up in 2 N NaOH and extracted 5×s with EtOAc. The aq. phase was placed on a rotary evaporator to remove the remaining EtOAc. The resulting clear solution was transferred to an erlenmeyer flask, cooled in an ice bath, and conc. HCl was added dropwise while stirring to pH 4. The resulting solid was collected by filtration providing 1.63 g of Intermediate R. The product was analyzed by chiral SFC (UV detection) using isocratic method (mobile phase: 25% MeOH with 0.1% DMEA, supercritical CO₂) on ChiralPak AD-H, 10×250 mm, 5 μm particle size, giving an enantiomeric purity of >95%, Rₜ 3.88 min (isomer 1) and 4.79 min (isomer 2). ¹H NMR (400 MHz, CDCl₃) δ ppm 1.37 (ddd, J=8.20, 6.64, 4.69 Hz, 1H), 1.67 (ddd, J=9.28, 5.08, 4.79 Hz, 1H), 1.87 (ddd, J=8.50, 4.69, 4.39 Hz, 1H), 2.48-2.63 (m, 1H), 6.87-7.06 (m, 2H), 7.37-7.46 (m, 2H).

1. A solid form comprising Form I of Compound I:

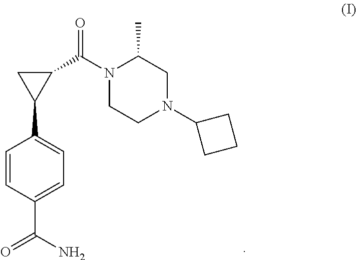

2. The solid form of claim 1, which has an XRPD pattern comprising at least one peak selected from about 5.3, about 8.5, and about 18.0 2θ, when measured using radiation with a wavelength of about 1.54 angstroms.

3. The solid form of claim 1, which has an XRPD pattern comprising at least two peaks selected from about 5.3, about 8.5, and about 18.0 2θ, when measured using radiation with a wavelength of about 1.54 angstroms.

4. The solid form of claim 1, which has an XRPD pattern comprising peaks at about 5.3, about 8.5, and about 18.0 2θ, when measured using radiation with a wavelength of about 1.54 angstroms.

5. The solid form of claim 2, wherein the pattern further comprises peaks at about 16.3 and about 19.3 2θ.

6. The solid form of claim 2, wherein the pattern further comprises peaks at about 20.9 and about 21.4 2θ.

7. The solid form of claim 1, which has an XRPD pattern comprising peaks at the following ten positions: about 5.3, about 8.5, about 10.6, about 15.5, about 16.3, about 18.0, about 18.4, about 19.3, about 20.9, about 21.4 2θ, when measured using radiation with a wavelength of about 1.54 angstroms.

8. The solid form of claim 1, which has an XRPD pattern essentially as depicted in FIG. 1.

9. The solid form of claim 1, which has a DSC thermogram essentially as depicted in FIG. 2.

10. The solid form of claim 1, which has a DSC thermogram comprising an endotherm at about 133.5° C.

11. The solid form of claim 1, which has a TGA thermogram essentially as depicted in FIG. 3.

12. The solid form of claim 1, which has a TGA thermogram comprising a mass loss of less than about 1% between about 20° C. and about 100° C.

13. The solid form of claim 12, wherein the mass loss is less than about 0.5%.

14. The solid form of claim 13, wherein the mass loss is about 0.25%.

15. The solid form of claim 1, which is substantially crystalline.

16. The solid form of claim 1, which is substantially pure.

17. The solid form of claim 1, which is substantially crystalline and substantially pure.

18. A pharmaceutical composition comprising the solid form of claim 1, in admixture with a pharmaceutically acceptable carrier or diluent.

19. (canceled)

20. (canceled)

21. (canceled)

22. A method for the treatment of a disorder selected from schizophrenia, narcolepsy, excessive daytime sleepiness, obesity, attention deficit hyperactivity disorder, pain, Alzheimer's disease, cognition deficiency, and cognition deficiency associated with schizophrenia, in a warm-blooded animal, comprising administering to said animal in need of such therapy a therapeutically effective amount of the solid form of claim 1.

23. A method for the treatment of a disorder selected from schizophrenia, narcolepsy, excessive daytime sleepiness, obesity, attention deficit hyperactivity disorder, pain, Alzheimer's disease, cognition deficiency, and cognition deficiency associated with schizophrenia, in a warm-blooded animal, comprising administering to said animal in need of such therapy a pharmaceutical composition according to claim 18.

24. (canceled)

25. (canceled)
